(19) United States
(12) Patent Application Publication
(10) Pub. No.: US 2024/0002803 A1
(43) Pub. Date: Jan. 4, 2024

(54) PLURIPOTENT STEM CELL AGGREGATES AND MICROTISSUES OBTAINED THEREFROM FOR THE CULTURED MEAT INDUSTRY

(71) Applicant: SuperMeat the Essence of Meat Ltd., Ness Ziona (IL)

(72) Inventors: Ido SAVIR, Herzliya (IL); Tomer HALEVY, Nahariya (IL); Yuval LEVY PERETZ, Rehovot (IL)

(73) Assignee: SuperMeat the Essence of Meat Ltd., Ness Ziona (IL)

(21) Appl. No.: 18/219,802

(22) Filed: Jul. 10, 2023

Related U.S. Application Data
(63) Continuation of application No. PCT/IL2022/050033, filed on Jan. 10, 2022.
(60) Provisional application No. 63/135,677, filed on Jan. 10, 2021.
(60) Provisional application No. 63/139,374, filed on Jan. 20, 2021.
(60) Provisional application No. 63/256,679, filed on Oct. 18, 2021.

(51) Int. Cl. C12N 5/077; C12N 5/00; A23L 33/17

(52) U.S. Cl. CPC C12N 5/0653; C12N 5/0658; C12N 5/0031; A23L 33/17; C12N 2533/54; C12N 2500/30

(57) ABSTRACT

Primed cells, pluripotent stem cell aggregates and microtissues produced therefrom and uses of same for producing edible products.

### RELATED APPLICATIONS

This application is a Continuation of PCT Patent Application No. PCT/IL2022/050033 having International filing date of Jan. 10, 2022, which claims the benefit of priority under 35 USC § 119(e) of U.S. Provisional Patent Application Nos. 63/135,677 filed on Jan. 10, 2021, 63/139,374 filed on Jan. 20, 2021, and 63/256,679 filed on Oct. 18, 2021. The contents of the above applications are all incorporated by reference as if fully set forth herein in their entirety.

### FIELD AND BACKGROUND OF THE INVENTION

The present invention, in some embodiments thereof, relates pluripotent stem cell aggregates and microtissues obtained therefrom for the cultured meat industry.

Successful production of cultured or as commonly referred to as clean meat, demands facing three major considerations: i. Cells origin ii. Culture conditions and iii. scalability.

i. Cells origin: Since their discovery, Embryonic Stem Cells (ESCs) have always inspired scientists due to their self-renewal capacity and their ability to differentiate virtually into any type of cell (6). Major progress in stem cell research has enabled the utilization of such cells in numerous scientific and medical applications and provided valuable data regarding optimization of necessary culture conditions (7). ESCs are derived from early embryos (e.g. the mammalian blastocyst or avian stage X embryos) and cultured indefinitely as long as their spontaneous differentiation is being prevented (8). This feature is particularly appealing to the cultured meat industry due to the massive number of cells needed for meeting with market demands. Following the growth phase, ESCs can be differentiated into specific lineages (9) in order to recapitulate the desired tastes and textures of specific animal tissues in the final product. While much of the aforementioned has been applied, mostly for academic and medicinal purposes, the cultured meat industry is still investing much resources in developing new sources and types of cell lines with stem cell characteristics such as self-renewal and differentiation potential with rapid doubling time. Another type of cells used for clean meat applications are primary and progenitors from adult tissues. While these cells still maintain some self-renewal capacity, their potency is somewhat limited to specific lineages (10).

ii. Culture conditions cultured cells are commonly grown in vitro in buffered media, which contain nutritional compounds (e.g. salts, fatty acids, amino acids, sugars, growth factors) and serum of animal origin (13). Most available media for vitro cell culture was originally optimized for small scale applications (14) and therefore other variables like financial costs, consumer sensitivity to animal ingredients, and ability of the medium to support dense cell populations were neglected.

iii. Scalability most if not all cells need to be adapted for highly dense, large volume cultures. The currently accepted methodology to achieve such cultures is by using suspension cultures in monitored bioreactors with constant stirring, encouraging cells to grow as aggregates as adherent cells on microcarriers in 2D (15). The most challenging part of this process however, would be scaling up differentiation or maturation of the cells with or without co-culturing, to produce a tissue like structure.

The case of cultured chicken cells is particularly appealing, as chicken meat is considered to be highly nutritional with a high proportion of protein to muscle/fat rate and chickens are the most consumed organism on the planet (16). For these reasons, clean sustainable alternatives for its production are most desirable. As used herein “cultured meat” is interchangeable with “clean meat”.

While being a significant model for embryonic development studies in the field of embryonic development, the case of avian embryonic stem cells seemed to attract much less attention compared to other models (e.g. Mouse/Human ESCs). Avian ESCs can be obtained from stage X-XIII embryonic disc (17). These blastodermal cells exhibit all features of stem cells including telomerase activity and expression of embryonic markers (e.g. alkaline phosphatase, SSEA1, Nanog, Oct4) (18). However, the easy access to avian blastodermal cells, alongside their ability to self-renew in culture led to their application in the animal vaccine industry (20). Similarly to the cultured meat industry, vaccine production in culture also demands the cultivation of cells in large dense populations. Several works have demonstrated recently the ability to adapt avian ESC cells from duck and chicken origin to suspension based, large volume culture conditions (21).

### SUMMARY OF THE INVENTION

According to an aspect of some embodiments of the present invention there is provided a method of producing pluripotent stem cell aggregates, the method comprising:
(a) providing a non-human pluripotent stem cells grown under conditions of matrix adherence;
(b) gradually depriving the non-human stem cell line of the matrix adherence, so as to obtain aggregates comprising the non-human pluripotent stem cells, the non-human pluripotent stem cells of the aggregates exhibiting a doubling time of no more than 12 hours in an undifferentiated manner for more than 60 passages, capable of differentiating into muscle, fat and connective tissue upon differentiation induction, exhibiting cell to cell adhesion lower than that of embryoid bodies (EBs) as determined by reduces expression of adhesion molecules selected from the group consisting of COL6A2, CD44, COL6A1, ANXA1, ANXA2 and S100A11, wherein steps (a) and (b) are performed in the presence of growth factors.

According to an aspect of some embodiments of the present invention there is provided a method of producing a microtissue comprising one or more cell types, the method comprising:
(a) forming in suspension, without matrix adherence, aggregates comprising non-human pluripotent stem cells, the non-human pluripotent stem cells of the aggregates exhibiting a doubling time of no more than 12 hours in an undifferentiated manner for more than 60 passages, capable of differentiating into muscle, fat and connective tissue upon differentiation induction, growing in the presence of growth factors, exhibiting cell to cell adhesion lower than that of embryoid bodies (EBs) as determined by reduces expression of adhesion molecules selected from the group consisting of COL6A2, CD44, COL6A1, ANXA1, ANXA2 and S100A11; and
(b) transferring the aggregates to a bioreactor for growth in suspension without matrix adherence in the absence of growth factors to thereby produce the microtissue.

According to an aspect of some embodiments of the present invention there is provided a method of producing a microtissue comprising one or more cell types of interest, the method comprising:
(a) producing the microtissue as described herein; and
(b) subjecting the microtissue to differentiation conditions in suspension to thereby produce the microtissue comprising one or more cell types of interest.

According to some embodiments of the invention, numerical values indicated are provided under optimal conditions for cell growth of a type and developmental stage as the non-human pluripotent stem cells.

According to some embodiments of the invention, the non-human stem cells are embryonic stem cells.

According to some embodiments of the invention, the embryonic stem cells are of an embryonic stem cell line.

According to some embodiments of the invention, the non-human pluripotent stem cells are of a livestock pluripotent stem cells.

According to some embodiments of the invention, the non-human pluripotent stem cells are selected from the group of avian pluripotent stem cells, bovine pluripotent stem cells, porcine pluripotent stem cells, goat pluripotent stem cells, sheep pluripotent stem cells, shrimp pluripotent stem cells and fish pluripotent stem cells.

According to some embodiments of the invention, the avian pluripotent stem cells are selected from the group of chicken pluripotent stem cells and duck pluripotent stem cells.

According to some embodiments of the invention, the avian pluripotent stem cells are chicken pluripotent stem cells.

According to some embodiments of the invention, the microtissue is 30-500 μm in diameter.

According to some embodiments of the invention, the microtissue is 30-500 μm in diameter.

According to some embodiments of the invention, the non-human pluripotent stem cells of the aggregates exhibit an average diameter of 80-120 μm.

According to some embodiments of the invention, the non-human pluripotent stem cells of the aggregates exhibit alkaline phosphatase expression.

According to some embodiments of the invention, the non-human pluripotent stem cells of the aggregates exhibit telomerase gene expression.

According to some embodiments of the invention, the non-human pluripotent stem cells of the aggregates being SSEA4−, LIN28+, ENS-1+, NANOG+, OCT4,+ and TRA-I-60+.

According to some embodiments of the invention, the aggregates or microtissue exhibit organoleptic properties of a native meat product.

According to some embodiments of the invention, the one of more tissue types are selected from the group consisting of a muscle cell, a fat cell and a connective tissue cell.

According to some embodiments of the invention, the matrix adherence is selected from feeder cells and a native or synthetic matrix molecule.

According to some embodiments of the invention, the matrix molecule comprises gelatin.

According to some embodiments of the invention, the growth factors are selected from the group consisting of IGF-1, IL6, sIL6 Rα, hLIF and stem cell factor (SCF).

According to some embodiments of the invention, the growth factors comprise IGF-1, IL6, sIL6 Rα, hLIF and stem cell factor (SCF).

According to some embodiments of the invention, each step of the method is devoid of animal components other than the non-human pluripotent stem cells.

According to some embodiments of the invention, the aggregates exhibit about the same gene expression as that of a stem cell line from which they are derived, excluding expression levels of cell motility and migration-related genes.

According to some embodiments of the invention, the microtissue is NANOG-OCT4−LIN28−, SSEA3+.

According to an aspect of some embodiments of the present invention there is provided a microtissue obtainable according to the method as described herein.

According to an aspect of some embodiments of the present invention there is provided a pluripotent stem cell aggregates obtainable according to the method as described herein.

According to an aspect of some embodiments of the present invention there is provided a microtissue comprising one or more cell types, the microtissue being 30-500 μm in diameter, wherein cells of the microtissue are NANOG-OCT4− LIN28−, SSEA3+.

According to some embodiments of the invention, the microtissue exhibits organoleptic properties of a native meat product.

According to some embodiments of the invention, the one or more cell types comprise a fat cell and a muscle cell.

According to an aspect of some embodiments of the present invention there is provided a pluripotent stem cell aggregate comprising non-human pluripotent stem cells, the non-human pluripotent stem cells of the aggregates exhibiting a doubling time of no more than 12 hours in an undifferentiated manner for more than 60 passages, capable of differentiating into muscle, fat and connective tissue upon differentiation induction and exhibiting cell to cell adhesion lower than that of embryoid bodies (EBs) as determined by reduced expression of adhesion molecules selected from the group consisting of COL6A2, CD44, COL6A1, ANXA1, ANXA2 and S100A11 as compared to the EBs.

According to some embodiments of the invention, the pluripotent stem cell aggregate exhibits at least one of:
(i) an average diameter of 80-120 μm;
(ii) the non-human pluripotent stem cells of the aggregate exhibit alkaline phosphatase expression;
(iii) the non-human pluripotent stem cells of the aggregate exhibit telomerase gene expression;
(iv) the non-human pluripotent stem cells of the aggregate are SSEA4−, LIN28+, ENS-1+, NANOG+, OCT4,+ and TRA-I-60+;
(v) exhibit organoleptic properties of a native meat product;
(vi) the aggregates exhibit about the same gene expression as that of a stem cell line from which they are derived, excluding expression levels of cell motility and migration-related genes.

According to some embodiments of the invention, the non-human pluripotent stem cells are selected from the group of avian pluripotent stem cells, bovine pluripotent stem cells, porcine pluripotent stem cells, goat pluripotent stem cells, sheep pluripotent stem cells, shrimp pluripotent stem cells and fish pluripotent stem cells.

According to some embodiments of the invention, the avian pluripotent stem cells are selected from the group of chicken pluripotent stem cells and duck pluripotent stem cells.

According to some embodiments of the invention, the avian pluripotent stem cells are chicken pluripotent stem cells.

According to an aspect of some embodiments of the present invention there is provided a food comprising the microtissue or aggregate as described herein.

According to an aspect of some embodiments of the present invention there is provided a method of producing food, the method comprising combining the microtissue or aggregate as described herein with an edible composition for human consumption.

According to an aspect of some embodiments of the present invention there is provided a method of producing cells, the method comprising culturing the stem cells having an adipocyte fate while retaining a proliferative phenotype in the presence of fatty acids at a concentration above 100 μM for a time sufficient to allow differentiation to adipocytes.

According to some embodiments of the invention, the culturing the stem cells having the adipocyte fate is effected for 5-10 days.

According to an aspect of some embodiments of the present invention there is provided cells obtainable according to the method as described herein.

According to an aspect of some embodiments of the present invention there is provided a microtissue obtainable according to the method as described herein.

According to an aspect of some embodiments of the present invention there is provided a pluripotent stem cell aggregates obtainable according to the method as described herein.

According to an aspect of some embodiments of the present invention there is provided a food comprising the cells as described herein or microtissue or aggregate as described herein.

According to an aspect of some embodiments of the present invention there is provided a method of producing food, the method comprising combining the cells or microtissue or aggregate of claim as described herein with an edible composition for human consumption.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIGS. 1A-E: Isolation of cESCs. a. Isolated cESCs plated on feeder cell layer generating cell clumps. b. Typical established stem cell colony grown with feeder cells. c. cESCs morphology plated without the addition of a feeder layer d. typical stem cell high nuclei/cytoplasm ration in proliferating cells. e. Proliferating colonies of cESCs on a feeder free culture dish.

FIGS. 2A-F: adaptation of cESCs to growth in suspension: a. EB morphology of a cESC colony moved to suspension. b. Single-cell morphology of cESC adapted to suspension. c-e .early staged aggregates of suspension adapted cESCs. f. mature cell aggregate of suspension adapted aggregate.

FIG. 3: Doubling time of aggregates cells growing in suspension. The graph demonstrates three separate experiments of aggregates suspension growth. Averaged doubling time was calculated for 75-120 hours runs. Calculated doubling time ranged between 10-12 hours with averaged doubling time of 11.9 hours per cycle.

FIG. 4: expression of stem cell markers in aggregates cells. Immunofluorescence staining revealed high and global expression of the stem cell markers Oct4, Nanog, in28, Tra-1-60 and ENS-1 (chicken stem cells marker). Surface marker SSEA3/4 found negative.

FIGS. 5A-C: self renewal of aggregates: a. aggregates stain positive to alkaline phosphatase.
b. high magnification of AP positive aggregate c. Representative example for telomerase activity in three populations of aggregates

FIG. 6: representative oncogenes and tumor suppressor genes expression level in aggregates samples compared to chicken cES. Analysis demonstrated aggregates retaining the expression level of oncogenes compared to cES. Tumor suppressors were either unchanged or elevated compared to chicken ES.

FIGS. 7A-B: Differentiation of aggregates cells into fat accumulating cells (A) a. Initial culture of undifferentiated aggregates. b. Mature aggregated of aggregates, three days in culture, prior to the addition of differentiation media. c. alteration of morphology of aggregates cells 4 days following treatment with fat differentiation media. d. Single aggregates cell 4 days after differentiation media treatment. Demonstrating the accumulation of droplets within the cell' cytoplasm. (B) validation of fat droplets accumulation in differentiated aggregates. a. Differentiated aggregates stained positive for BODIPY staining, evidence for fat accumulation. b. High magnification of differentiated, fat accumulating cells.

FIG. 8: differentiation of aggregates into fibroblasts. Following treatments aggregates adhered to plate surface (upper left) followed by disintegration of aggregates (upper right), resulting in adoption of single layer epithelial fibroblast cells.

FIG. 9: rhythmic contractions of aggregates undergo muscle differentiation. Contractions occur once in 1.5-2 sec.

FIG. 10: the culture of microtissues in serum-free defined media. A representative example of the ability to adapt cells to various culture conditions. The presented clones cultured and propagated in GRO-I® (Merck) media supplemented with Ex-Cell® (Merck) chemically defined hydrolysates.

FIG. 11 shows expression of stem cell and ECM markers in micro-tissues. Oct4, Nanog and Lin28 is lost due to the differentiation process. However, cells acquired expression of SSEA3 (upper panel). In addition, enhanced expression of ECM and collagen family members in microtissues is found positive for the high presence of Col2A1 and Col9a2, as well as ECM markers Laminin and HSPG.

FIGS. 12A-E: muscle differentiation within defined regions of microtissues a. naïve aggregates in suspension growth. b. transfer of aggregates into differentiation conditions results in the emergence of cell protrusions in a reproducible manner. c &d. onset of muscle differentiation (indicated by MyHC staining) e. Formation of muscle differentiation regions in several locations of microtissue.

FIG. 13A-B: Expression of muscle progenitor markers in avian stem cells micro-tissues cultured in suspension.

FIGS. 14A-C: A. Formation of microtissues within suspended floating aggregates culture. Protrusions emerge from aggregates with elevated expression MyHC (Myosin heavy chain) indicating defined muscle identity. B. Maturation of muscle fibers differentiated from microtissues in adherent conditions. C. higher magnification of B.

FIGS. 15A-B: A. Differentiation of microtissues into mature muscle cells. (a)-(c): Expression of Troponin-T indicates commitment into cardiomyocyte lineage. B: Transfer of cells into adherence conditions encourage maturation and elongation of muscle fibers.

FIG. 16: Massive accumulation of fat droplets within differentiated microtissues into fat cells Following exposure to high doses of Oleic acid (upper panels) and Oleic acid and Linoleic acid combined treatment (lower panels).

FIGS. 17A-B: Differentiation of micro-tissues into mutually exclusive populations of fat and muscle cells. (a) Troponin-T staining emphasizes differentiation into muscle cells at the microtissue periphery while bodipy staining demonstrates the formation of fat accumulating cell populations within the microtissue core. The two populations are mutually exclusive. (b) Magnification of the squared region in panel a.

FIGS. 18A-E: parallel differentiation of microtissues into distinct fat and muscle cell populations: (a) typical arrangement of muscle cytoskeletal muscle within microtissues populations (Phalloidin staining). (b) nuclear staining of muscle fibers emphasizes the formation of multinucleated fibers by microtissues cells. (c). Typical arrangement of fat accumulating cells (adipocytes) microtissues populations (phalloidin staining) (d). Lipid staining (BODIPY) growth emphasizes fat accumulation within differentiating microtissues. (e). Parallel differentiation of microtissues cells into fat and muscle within the same culture emphasizes that fat and muscle cells originate from mutually exclusive populations.

FIG. 19 shows a schematic illustration of a process according to some embodiments of the invention. Embodiments of the invention also refer to portions of the process, e.g., 1-7, 8b-9a, 9b-a, 8b, 9b and the like.

### DESCRIPTION OF SPECIFIC EMBODIMENTS OF THE INVENTION

The present invention, in some embodiments thereof, relates to methods of producing microtissues for the cultured meat industry.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The self-renewal capacity of embryonic stem cells (ESCs) and chicken ESCs in particular is highly applicable for the cultured meat industry.

The novel platform is designed for optimal meat production. The platform provides several unique characteristics that position it well to serve as a strong industrial manufacturing tool for the meat and food industry being an inexhaustible non genetically modified (GMO) production Source. The cells have been shown to be naturally immortal over 100 passages, retaining stable and consistent growth and characteristics. This relieves the need to replenish the animal cell bank, removing the animal from the production process completely. The platform is based on the addition of xeno-free factors, rendering it completely devoid of animal components other than the cells themselves. The platform is highly proliferative in nature supporting a continuous production process allowing for a cost efficient and high yield manufacturing process.

Efficient Production Platform—the aggregates and microtissue lines demonstrate rapid self renewal capabilities and were able to reach doubling time as fast as 8 hours. Cell densities in these cultures exceed 10\hat{ }8 cells/ml. The lines retain high plasticity supporting a maturation process forming natural microtissue structures expressing a heterogeneous composition of fat, muscle and connective tissue cells in a controllable manner. Industry standard tests showed high similarity between the nutritional composition and profile of cultured and conventionally produced chicken meat. The platform produces raw material or can be used to compose products having organoleptic properties comparable to conventionally produced meat. Industry standard assays as well as multiple tasting panels show high similarity to conventionally produced chicken meat in terms of flavor profile and consumer experience.

Thus, according to an aspect of the invention there is provided a method of producing pluripotent stem cell aggregates, the method comprising:
(a) providing a non-human pluripotent stem cell line of non-human pluripotent stem cells grown under conditions of matrix adherence;
(b) gradually depriving the non-human stem cell line of the matrix adherence, so as to obtain aggregates comprising the non-human pluripotent stem cells, the non-human pluripotent stem cells of the aggregates exhibiting a doubling time of no more than 12 hours in an undifferentiated manner for more than 60 passages, capable of differentiating into muscle, fat and connective tissue upon differentiation induction, exhibiting cell to cell adhesion lower than that of embryoid bodies (EBs) as determined by reduced expression of adhesion molecules selected from the group consisting of COL6A2, CD44, COL6A1, ANXA1, ANXA2 and S100A11 as compared to the EBs, wherein steps (a) and (b) are performed in the presence of growth factors.

As used herein “pluripotent stem cells” refers to non-human cells which can differentiate into all three embryonic germ layers, i.e., ectoderm, endoderm and mesoderm or remaining in an undifferentiated state. The pluripotent stem cells include embryonic stem cells (ESCs) and induced pluripotent stem cells (iPS).

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus any time during gestation, and cells originating from an unfertilized ova which are stimulated by parthenogenesis (parthenotes).

The main source for Avian embryonic stem cells is a fertilized unincubated egg (Day 0). At this stage the embryo consists of 60-100K pluripotent cell locked in arrest state. The arrest phase is crucial in order to allow the hen to synchronize the hatching of several eggs that was being laid in different days. Propagation of these cells in-vitro occurs upon incubation in 39° C. (e.g., Pokharel, N et al. Poult Sci. 2017 Dec. 1; 96(12):4399-4408. doi: 10.3382/ps/pex242. PMID: 29053871).

Induced pluripotent stem cells (iPS; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin) and undergo de-differentiation by genetic manipulation which re-program the cell to acquire embryonic stem cells characteristics. According to some embodiments of the invention, the induced pluripotent stem cells are formed by inducing the expression of Oct-4, Sox2, Kfl4 and c-Myc in a somatic stem cell.

As used herein “an aggregate” refers to a group of proliferative, non-differentiated i.e., pluripotent cells that are bound to each other via secretion of adhesive molecules (e.g. ECM). The size can be between 30-1000 mm e.g., 50-500 um, e.g., 30-100 um, 100-500 um, 100-400 um, 200-500 um, 300-400 um, 100-200 um, 50-200 um, 500-1000 um, 700-1000 um. It will be appreciated that where size is indicated throughout the document, the size refers to an average size in a population of aggregates or microtissues.

Markers: Oct4+, Lin28+, SSEA1+, SSEA4−, ENS1+, Tra-1-60+. nanog+. According to a specific embodiment, the markers are: Oct4+, Lin28+, SSEA4−, ENS1+, Tra-1-60+. Nanog+.

According to a specific embodiment, the aggregate is grown in the presence of growth factors (e.g., IGF1, SCF, IL6, IL6Rα, LIF hLIF combinations thereof, or additionally or alternatively, IWR1, FGF2 or others) or signaling inhibitors such as inhibitors of Rho (e.g., Y27632) MEK, GSK3, FGFR3, N2B27-3, or as further exemplified below.

According to a specific embodiment, about 90-100% of the cells in the aggregates are pluripotent stem cells.

As mentioned, the cells are non-human pluripotent stem cells.

According to a specific embodiment, the non-human pluripotent stem cells are of a livestock pluripotent stem cells.

According to a specific embodiment, the non-human pluripotent stem cells are selected from the group of avian pluripotent stem cells, bovine pluripotent stem cells, porcine pluripotent stem cells, goat pluripotent stem cells, sheep pluripotent stem cells, shrimp pluripotent stem cells and fish pluripotent stem cells.

As used herein, the term “avian” to any species, subspecies or race of organism of the taxonomic Class Ayes, such as, but not limited to, such organisms as chicken, turkey, duck, goose, quail, pheasants, parrots, finches, hawks, crows and ratites including ostrich, emu and cassowary. The term includes the various known strains of Gallus gallus (chickens), for example, White Leghorn, Brown Leghorn, Barred-Rock, Sussex, New Hampshire, Rhode Island, Australorp, Cornish, Minorca, Amrox, California Gray, Italian Partidge-coloured, as well as strains of turkeys, pheasants, quails, duck, game hen, guinea fowl, squab, ostriches and other poultry commonly bred in commercial quantities.

In a specific embodiment, the avian cells are chicken cells.

In one example, the cells are from avian embryonic-derived stem cell line EB14 (chicken) or EB66 (duck) (WO2005042728).

According to an embodiment, the cells throughout the methods are non-genetically modified.

In one embodiment, the pluripotent stem cells are of a stem cell line.

Pluripotent stem cells are adherent by nature and hence are grown under conditions of cells adherence, also referred to as a two dimensional culture (2D).

According to specific embodiments, a 2D culture relates to growth on a two-dimensional matrix (feeder layer-free) or on feeder cells.

Thus, the pluripotent stem cells can be grown (expanded) on a solid surface such as an extracellular matrix (e.g., gelatin, fibronectin, Matrigel® or laminin) in the presence of a culture medium.

According to a specific embodiment, the surface is gelatin.

According to a specific embodiment, the cells are grown on a feeder layer.

According to a specific embodiment, the feeder cells are mouse embryonic fibroblasts (MEFS).

According to a specific embodiment, the cells are cultured in the presence of factors and optionally serum (e.g., fetal bovine serum, horse serum, and/or fish serum from trout) or serum replacement (e.g., yeast or plant hydrolysates e.g., soy. Other factors may be included at this stage e.g., Na-pyruvate, Na-selenite, amino acids, 2-mercaptoethanol. Cells are allowed to propagate and passaged every 24-72 hrs.

According to a specific embodiment, the growth factor is selected from the group consisting of IGF-1, IL6, sIL6 Rα, hLIF and stem cell factor (SCF).

According to a specific embodiment, the growth factors comprise IGF-1, IL6, sIL6 Rα, hLIF and stem cell factor (SCF).

According to some embodiments of the invention, when the cells reach 3-5 passages they are gradually deprived from substance adherence (e.g., feeder layer) and grown for several passages to select for stable feeder-free clones. According to some embodiments, factors are still present at this stage.

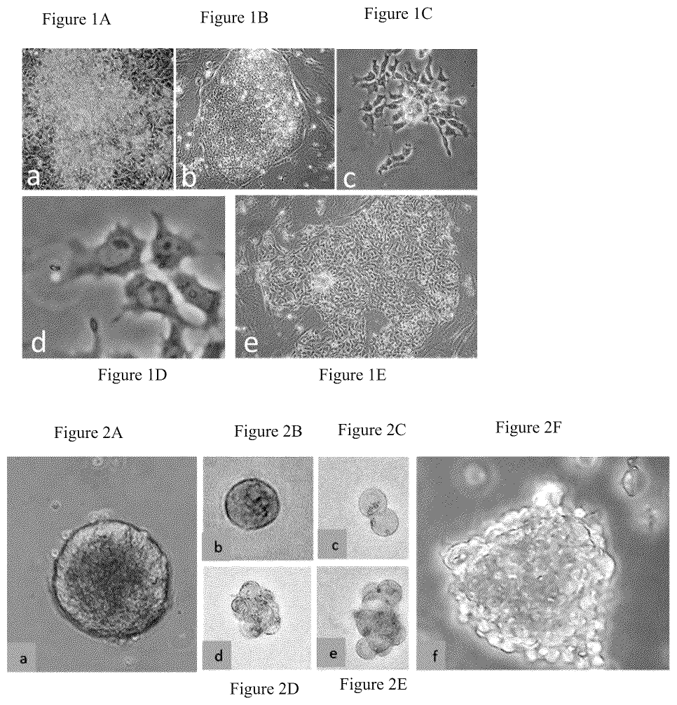

Notably, after the feeder layer withdrawal phase, the cells tend to form less compact stem cell colonies composed of large nucleated cells as they are not constrained by fibrous cells (FIGS. 1c-e). According to a specific embodiment, at this stage, cells exhibit the expected doubling time of about 24 hours per cycle. This stage is also referred to as “gradually depriving the non-human stem cell line of the matrix adherence”.

As used herein “gradually depriving” refers to deprivation from matrix adherence (not from GFs).

To do this, cells are gradually adapted to grow in suspension rather than as adherent cells.

As used herein the phrase “suspension culture” refers to a culture in which the pluripotent stem cells are suspended in a medium rather than adhering to a surface.

Thus, the culture of the present invention is “devoid of matrix adherence” in which the pluripotent stem cells are capable of expanding without adherence to an external substrate such as components of extracellular matrix, a glass microcarrier or beads.

According to some embodiments, cells are gradually displaced from adhesive surfaces (such as those comprising an adhesive matrix e.g., gelatin, laminin, fibronectin, poly-L-lysine) and subtle shaking is imposed (e.g., 50-100 rpm) and optionally mechanical dissociation of the aggregates. Shaking may be gradually increased at every passage. According to a specific embodiment, by the continuous selection for a period of about 2-3 months, cells are encouraged to down-regulate different adhesion molecules while expressing others, allowing over time the formation of 3D loose raspberry-like aggregates, with a clear definition of each cell composing the aggregate, as opposed to a structure of an embryoid body. At this stage, cells have become very stable, and doubling time is reduced to between 10-20, e.g., 18-20 h, 10-12, h, 12-14 h, 12-16 h, 10-16 h, 12-18 h per cycle such as in the case of avian embryonic stem cells.

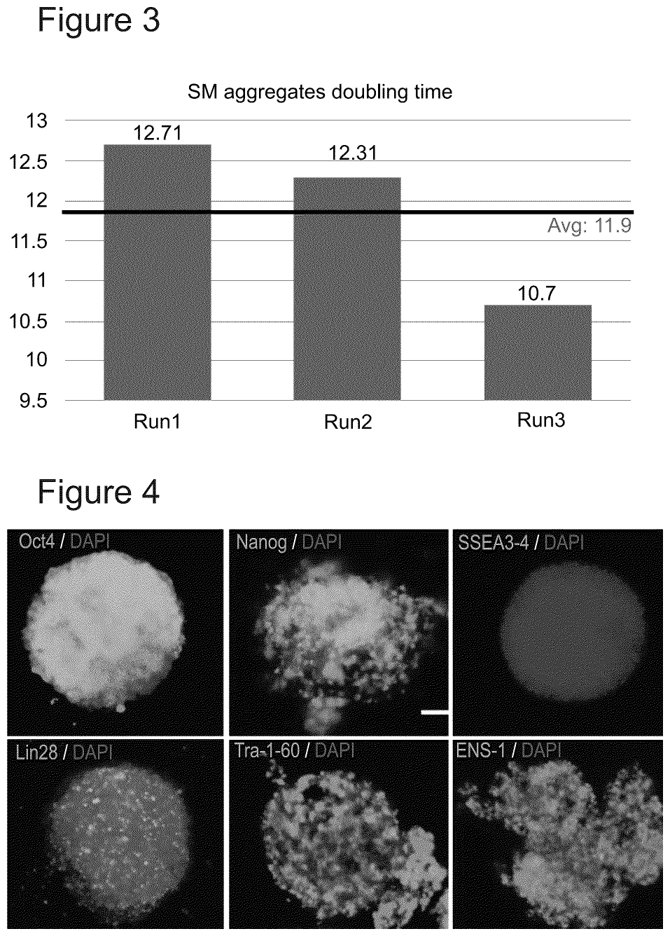

According to a specific embodiment, the aggregate are of an aggregate forming cell line. Thus, following the adaptation of cells to growth in suspension the cells are adapted to continued rapid growth in a reproducible manner such as in a stirred bioreactor environment to ensure the ability of the cells to be suitable for industrial scale-up. For this, clones are tested for the generation of a cell line that grows as aggregated cells, with a high proliferative rate, optionally in high-velocity stirring (200-400 rpm tip speed) in stirred bioreactors, while maintaining the aggregate's integrity and stem cell characteristics. Following this process of optimization, several cell lines are produced with all favorable characteristics, these cells are named “SMCMC”, according to some embodiments of the invention. These cells exhibit the desired morphology, differentiation potential, and a doubling time of 10-12 hours per cycle, with some growing conditions showing 8 hours per cycle. (FIG. 3).

Such aggregate forming cell lines can be stored in a cell bank.

According to an aspect of the invention there are provided pluripotent stem cell aggregates.

According to a specific embodiment, the aggregates are obtainable according to the methods as described herein.

According to a specific embodiment, the aggregates exhibit an average diameter of 80-120 μm.

According to a specific embodiment, the non-human pluripotent stem cells of the aggregates exhibit alkaline phosphatase expression.

According to a specific embodiment, the non-human pluripotent stem cells of the aggregates exhibit telomerase gene expression.

According to a specific embodiment, the non-human pluripotent stem cells of the aggregates are SSEA4−, LIN28+, ENS-1+, NANOG+, OCT4,+ and TRA-I-60+, such as determined at the RNA level.

According to a specific embodiment, the hon-human pluripotent stem cells of the aggregates do not display oncogenic transformation.

According to an aspect of the invention there is provided a pluripotent stem cell aggregate comprising non-human pluripotent stem cells, the non-human pluripotent stem cells of the aggregates exhibiting a doubling time of no more than 12 hours in an undifferentiated manner for more than 60 passages, capable of differentiating into muscle, fat and connective tissue upon differentiation induction and exhibiting cell to cell adhesion lower than that of embryoid bodies (EBs) as determined by reduced expression of adhesion molecules selected from the group consisting of COL6A2, CD44, COL6A1, ANXA1, ANXA2 and S100A11 as compared to the EBs.

According to a specific embodiment, the pluripotent stem cell aggregate exhibits at least one of:
(i) an average diameter of 80-120 μm;
(ii) the non-human pluripotent stem cells of the aggregate exhibit alkaline phosphatase expression;
(iii) the non-human pluripotent stem cells of the aggregate exhibit telomerase gene expression; (iv) the non-human pluripotent stem cells of the aggregate are SSEA4−, LIN28+, ENS-1+, NANOG+, OCT4,+ and TRA-I-60+;
(v) exhibit organoleptic properties of a native meat product;
(vi) the aggregates exhibit about the same gene expression as that of a stem cell line from which they are derived, excluding expression levels of cell motility and migration-related genes.

According to a specific embodiment, the aggregate exhibits a combination of i+ii. I+ii+iii, i-iv, i-v, i-vi, ii-iii, ii-iv, ii-v, ii-vi, ii-v, iii-vi, iii-vi, iv-v, iv-vi, v-vi.

According to a specific embodiment, the non-human pluripotent stem cells are selected from the group of avian pluripotent stem cells, bovine pluripotent stem cells, porcine pluripotent stem cells, goat pluripotent stem cells, sheep pluripotent stem cells, shrimp pluripotent stem cells and fish pluripotent stem cells.

According to a specific embodiment, the avian pluripotent stem cells are selected from the group of chicken pluripotent stem cells and duck pluripotent stem cells.

According to a specific embodiment, the avian pluripotent stem cells are chicken pluripotent stem cells.

Exemplary aggregates according to some embodiments of the invention are shown in FIGS. 4-6 (avian e.g., chicken).

According to a specific embodiment, the aggregates exhibit about the same gene expression as that of a stem cell line from which they are derived, excluding expression levels of cell motility and migration-related genes, such as determined at the RNA level (see Examples section) or protein level (e.g., immunostaining).

According to a specific embodiment, the cells of the aggregates and microtissues keep a normal karyotype.

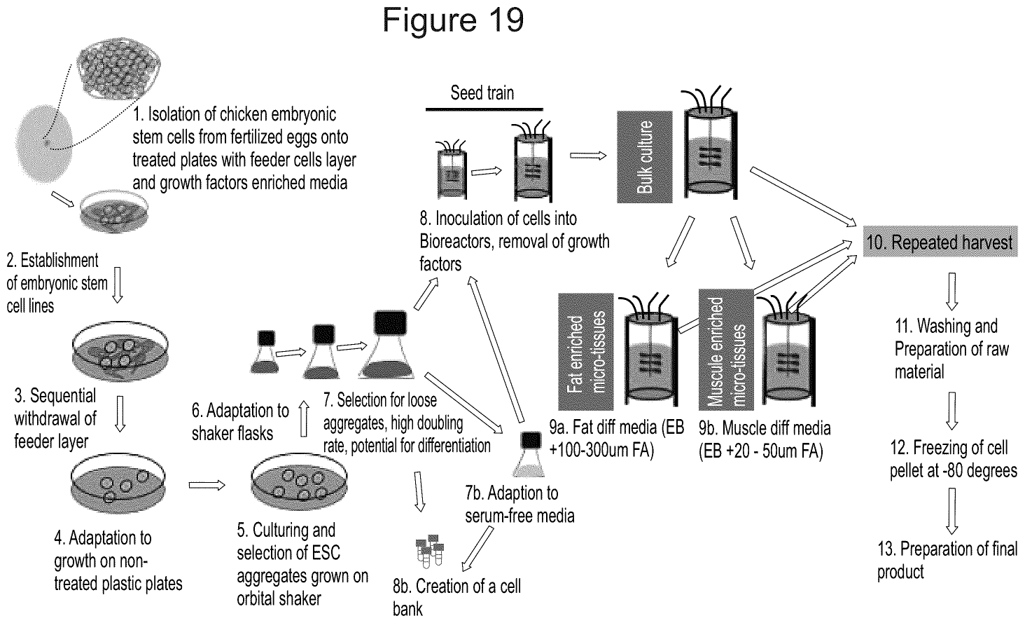

According to a specific embodiment, the above covers steps 1-7 of FIG. 19. According to a specific embodiment these steps are performed in the presence of serum, although as mentioned serum can be replaced by a serum replacement or other substitutes such as yeast or plant hydrolysates.

Cultured meat production puts a special emphasis on the media being used to culture the cells. Tissue culture media are traditionally designed for other purposes (e.g. research, pharmaceutical, clinical production and more) and therefore carry several caveats from the perspective of culture meat production. The cost of the culture media is generally the biggest economic burden on cultured meat production. Furthermore, most culture media available contain fetal calf (or other animal originated) serum (FCS). The use of FCS harbors the inherent flaw of being undefined (in terms of chemical composition) with high variability between batches. Moreover, the use of such material in culture meat production will most likely alienate public acceptance due to the problematic way it is produced. In order to overcome these obstacles the present inventors set out to adapt the aggregates and microtissues (as further described hereinbelow) to grow in serum free media, without compromising the quality, safety, and the unique characteristics of the microtissues.

FIG. 19 shows such an option.

In order to achieve this, microtissues (as further described below) or post formed aggregates (in shaking conditions) are adapted to grow in serum free media.

The adaptation to serum free media takes place following the propagation of ES cells as aggregates in shaking flasks (FIG. 19, stage 7b) either by additional propagation in flasks using serum free medium (FIG. 19 7b) and then transferring to stirred bioreactor (FIG. 19 stage 8b) or by direct seeding in bioreactor using serum free media (FIG. 19, stage 8b). The adaptation to serum free media is done by collecting highly proliferative aggregates populations and introducing them to serum—free media (FIG. 19, 7b). Gradual adaptation is carried out by reducing the percentage of serum e.g., FBS within the serum free media over time (e.g., 5% to 2.5% to 1% until complete withdrawal). In most cases the gradual adaptation duration is between 2-4 weeks. Another possibility to generate serum-free (SF) culture for high scale production is to transfer the aggregates directly to the serum free media. In this procedure, cells with the ability to survive and quickly adapt to the changed environment are collected. The length of such process is about 2 weeks. Either possibilities is concluded by the generation of cell bank for future use (FIG. 19 step 8a), according to some embodiments of the invention.

Thus as shown below, about 14 days the cells were adapted to the new media in the absence of serum and then the original 12 hours doubling time is restored and retained a high level of cell viability (FIG. 10). In a similar manner the present inventors were also able to adapt the cells to grow in other combinations of basal media supplemented with yeast, plant peptones and hydrolysates. Among the mixes which were used successfully for adaptation of cells to serum-free media, the present inventors could identify: DMEM (high glucose) supplemented with the Ex-Cell lysate, DMEM/F12 supplemented with Ex-Cell lysates, DMEM (low glucose) supplemented with Ex-Cell lysate, DMEM (high glucose) supplemented with combination of soy and yeast lysates manufactured by KERRY group. Lysates that tested successfully in this process were either a combination of all or part of these four products as follows: Hypep 1510 (ID: S-2048780, Item: U1-5X99023), SHEFF-VAX PLUS ACF(ID S-2048778, U1-5X00484.K1G), SHEFF-VAX PF ACF (ID:S-2048777, U1-5X01143.K1G), SHEFF-VAX PLUS PF ACF VP (ID: S-2048776, U1-5X01090). The present inventors also successfully adapted microtissues to combinations of routinely used basal media (DMEM HG/LG, DMEM/F12, RPMI 1640) with soy and yeast hydrolases manufactured by FUJIFILM-IRVINE Scientific (Ultrafiltered Soy Hydrolysate #IR-96857E; Ultrafiltered Yeast Hydrolysate #IR-96863E). In addition, microtissues were also adapted to grow in basal media (DMEM HG/LG, DMEM/F12, RPMI 1640) supplemented with different combinations of DIFCO's Select Soytone (#15ABP196), Bacto's Yeast Extract (#15ABP197), BBL's Phytone peptone (#15ABP195), BBL's Yeast extract (#15ABP202), Bacto Malt Extract (15ABP201) and Bacto TC yeastolate (#15ABP163). For this purpose, yeast originated lysates were combined with either one or several plant originated lysates. In all experiments the media also supplemented with Ethanolamine (20 ng/L), Insulin (100 ug/L), Selenium (50 ng/L) and Transferrin (55 ug/L). Media also supplemented with 1×MEM NEAA (Biological industries, 01-340-1B), 2 mM L-Alanine/L-Glutamine (Biological industries-03-022-1B) and 1 mM Sodium pyruvate (Biological industries). The results presented herein emphasize the ability of microtissues to adapt multiple combinations based on basal media supplemented with soy and plant hydrolysates as a substitution to serum in cultured media.

It will be appreciated that not all the steps are necessary to perform embodiments of the invention. Thus for instance, an aggregate may be provided from a cell bank or a commercial vendor and further used for the food industry as described below.

Hence, regardless of production, the aggregates can be used per se in food production or further subjected to a further development process for the generation of microtissues.

Thus, according to an aspect of the invention, there is provided a method of producing a microtissue comprising one or more cell types, the method comprising:
(a) forming in suspension, without matrix adherence, aggregates comprising non-human pluripotent stem cells, the non-human pluripotent stem cells of the aggregates exhibiting a doubling time of no more than 12 hours in an undifferentiated manner for more than 60 passages, capable of differentiating into muscle, fat and connective tissue upon differentiation induction, growing in the presence of growth factors, exhibiting cell to cell adhesion lower than that of embryoid bodies (EBs) as determined by reduced expression of adhesion molecules selected from the group consisting of COL6A2, CD44, COL6A1, ANXA1, ANXA2 and S100A11 as compared to the EBs; and
(b) transferring the aggregates to a bioreactor for growth in suspension without matrix adherence in the absence of growth factors to thereby produce the microtissue.

According to an alternative aspect there is provided a method of producing a microtissue comprising one or more cell types, the method comprising:
(a) providing a suspension culture which comprises aggregates comprising non-human pluripotent stem cells, the non-human pluripotent stem cells of the aggregates exhibiting a doubling time of no more than 12 hours in an undifferentiated manner for more than 60 passages, capable of differentiating into muscle, fat and connective tissue upon differentiation induction, growing in the presence of growth factors, exhibiting cell to cell adhesion lower than that of embryoid bodies (EBs) as determined by reduced expression of adhesion molecules selected from the group consisting of COL6A2, CD44, COL6A1, ANXA1, ANXA2 and S100A11 as compared to the EBs; and
(b) transferring the aggregates to a bioreactor for growth in suspension without matrix adherence in the absence of growth factors to thereby produce the microtissue.

At this stage the aggregates are transferred to a bioreactor in the absence of the growth factor, e.g., as described above.

As used herein bioreactor refers to a vessel, device or system designed to grow cells, aggregates or tissues in the context of cell culture. Such bioreactors are described by Popovic et al. BIOTECHNOLOGY—Bioreactoes and Cultivation Systems for Cell and Tissue Culture—M. K.

Popovic, Ralf Portner Encyclopedia of Life Support Systems (EOLSS) and further hereinbelow and in the Examples section which follows.

Selection of culture apparatus for production is based on the scale. Large-scale production preferably involves the use of dedicated devices. Continuous cell culture systems are reviewed in Furey (2000) Genetic Eng. News 20:10. Suitable bioreactors which can be used according to the present teachings include, but are not limited to, Sartorius Biostat STR, Sartorius Biostat BDCU, Thermo Fisher HyPerforma DynaDrive S.U.B., Eppendorf Bioflo 510.

Such systems can also be used for the production of the microtissues when they are used in the food industry.

As used herein the term “microtissues” refers to mesodermal cells which are grown in the absence of growth factors or signaling inhibitors are either non-fully or terminally differentiated.

As used herein “in the absence of growth factors” refers to a maturation medium which contains no added growth factors (on top of those in serum if present), causing the cells to lose key pluripotent markers, while gaining mesodermal properties, including specific markers, and elevated expression of connective tissue proteins, as compared for instance to cell aggregates of step 7 of FIG. 19. Due to their early differentiation stage and specific culturing conditions in the bioreactor, these microtissues are still able to proliferate rapidly and extensively and be expanded for at least 150 population doublings.

According to a specific embodiment, growth in the bioreactor is controlled by defined set-points of the bioreactor run parameters, such as agitation inside the bioreactor and gassing with tip speed ranging from 0.25-1.70 m/sec with optimal tip speed of 0.85 m/sec. These parameters in turn control the size of the microtissues. The size of the microtissue is important for the ability to differentiate a plurality of cell types in a single microtissue.

According to a specific embodiment, the microtissues are grown in a serum-free medium, as described hereinabove.

According to other embodiments, the microtissues are grown in the presence of serum.

This step of maturation (step 8 of FIG. 19) can last for extended periods of times such as above 150 passages.

As used herein “maturation” refers to the step that includes withdrawal of GFs (i.e., absence of growth factors, also referred to as “factor-free”) that allow pluripotent stem cells to begin mesodermal lineage commitment.

Various maturation protocols can be employed some are exemplified in the Example section which follows. For example, maturation of bESCs in suspension in monitored stirring bioreactor in factor free media (DMEM (HG)/10-20% FBS, 10 U/1 U, Penicillin/Streptomycin, 2 mM glutamine; or maturation of pESCs in suspension in stirring bioreactor conducted in factor free media (DMEM (HG)/10-20% FBS, 10 U/1 U, Penicillin/Streptomycin, 2 mM glutamine.

The step which does not include presence of growth factors can last from between 7 days to up to 12 months for instance, during which cells can be expanded harvested, stored or further differentiated to cells of specific interest (e.g., muscle, fat or combinations thereof).

Hence, there is provided a microtissue.

Thus, according to an aspect of the invention, there is provided a microtissue comprising one or more cell types, the microtissue being 30-500 μm in diameter, wherein cells (about 90%) of the microtissue are NANOG-OCT4− LIN28−, SSEA3+.

According to some embodiments of the invention, the microtissue exhibits organoleptic properties of a native meat product

According to some embodiments of the invention, the one or more cell types comprise a fat cell and a muscle cell.

When not subjected to specific differentiation protocol the microtissue is a non-fully differentiated microtissue which is not pluripotent.

As used herein “a non-fully differentiated microtissue” refers to an aggregate of cells comprising mesodermal lineage multipotent cells i.e., cells with the ability to develop into different types of mesodermal tissues. The size of the microtissue can be between 30-1000 μm, e.g., 50-300 uM, markers: Pax3+, Pax7+, MyoD+, SSEA4+, SSEA1−, oct4−, as determined at the RNA level (see e.g., Examples section). According to a specific embodiment, the aggregates of step 7 are smaller than the microtissue of steps 8b-9b of FIG. 19.

Such microtissues can be integrated in the food industry per se, or further subjected to a differentiation process.

Non-fully differentiated microtissues can be harvested therefore at this stage which is covered by step 8b of FIG. 19 describing embodiments of the invention in a non-limiting fashion.

Alternatively, these microtissues can be further subjected to differentiation.

Thus, according to an aspect of the invention there is provided a method of producing a microtissue comprising one or more cell types of interest, the method comprising:
(a) producing the microtissue (non-fully differentiated); and
(b) subjecting the microtissue to differentiation conditions in suspension to thereby produce the microtissue comprising one or more cell types of interest.

Specific protocols for differentiation are provided in the Examples section which follows, as well as in WO2018/189738, incorporated by reference in its entirety.

According to a specific embodiment, the differentiated microtissue is NANOG-OCT4-LIN28−, SSEA3+.

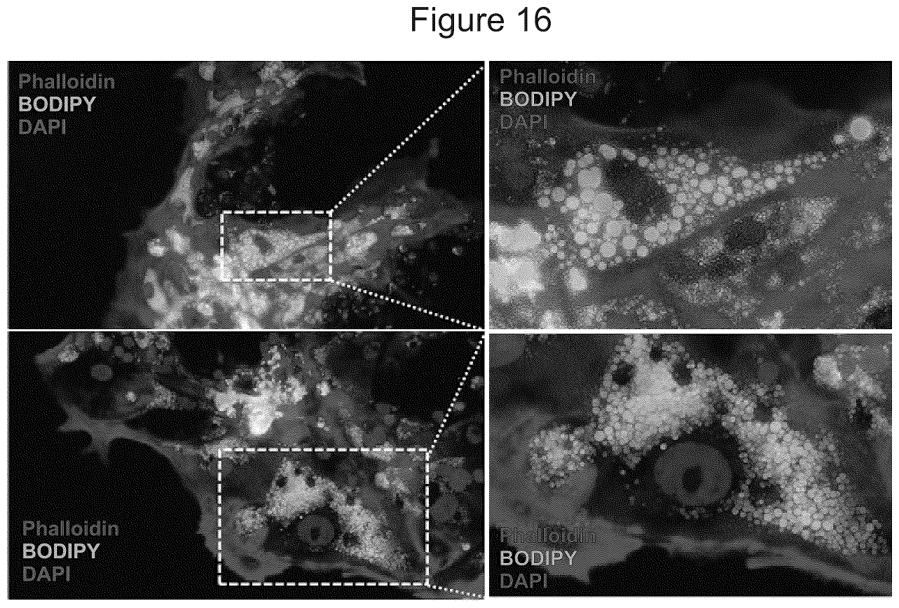

Hence “a differentiated microtissue” refers to an aggregate of cells composed of cells that underwent differentiation into mature muscles (TroponinT+, Myosin+), adipocytes (positive to oil-red and Bodipy staining), and collagen secreting cells (COL9+ COL1A+, COL2A+). The size can be between 30-5000 μm. See for instance FIGS. 16-18A-E as an exemplary embodiment.

According to a specific embodiment, the microtissue is 30-500 μm in diameter.

As mentioned, maturated microtissues or aggregates can be the subject to differentiation using differentiation protocols which are well known in the art.

Alternatively, the differentiation protocol of a microtissue or aggregate can be a multistep process.

Thus, according to an aspect of the invention, there is provided a method of cell priming towards an adipocyte fate, the method comprising culturing pluripotent or multipotent stem cells in the presence of fatty acids at a concentration not exceeding 100 μM for a time sufficient to obtain muscle cells and stem cells having an adipocyte fate while retaining a proliferative phenotype.

As used herein “adipocyte fate” refers to “pre-adipocytes” refers to mesenchymal stem cells that adopted commitment to adipogenic fate prior to their terminal differentiation. Pre-adipocytes tend to express Sca-1, cd-90, cd29. Pre-adipocytes also adopt fibroblastic morphology and demonstrate positive response to BODIPY staining, usually around the nuclei.

As used herein “priming” refers to achieving an adipocyte fate which is done by culturing in the presence of FAs (e.g., oleic acid, palmitic acid) or precursor thereof in low doses (e.g., below 100 uM e.g., 30-50 uM, 30-80 uM, 10-90 uM) to direct cell to adipocyte lineage.

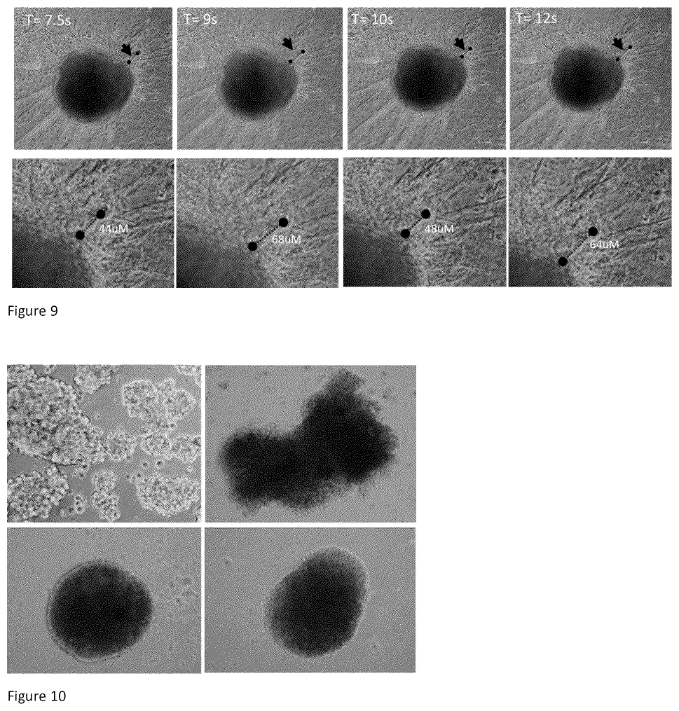

In the presence of low levels of fatty acids the cells i.e., below 100 uM e.g., 30-50 uM, 30-80 uM, 10-90 uM, the cells will acquire a muscle phenotype as can be seen in FIG. 9. However, increasing the levels, i.e., 100 uM or above, will drive the cells towards adipocytes.

‘Priming’ step: priming step includes adjusting the (e.g., avian) stem cells to growth in the presence of low dosages of different fatty acids. In this process, the present inventors add a certain or several fatty acids to the cell's media in a final concentration that does not exceed is below 100 uM (e.g., 10-100 uM or 10-50 uM).

Differentiation step: priming cells to adipogenic differentiation is followed by a terminal differentiation. Differentiating cells includes elevating the levels of FA e.g., 100 uM or above 100 uM, e.g., to 500 uM, e.g., for up to 14 days, e.g., 1-7 days, 1-10 days, 5-10 days.

According to a specific embodiment, the multipotent stem cells are adult stem cells.

According to a specific embodiment, the adult stem cells are mesodermal.

According to a specific embodiment, the mesodermal stem cells are mesenchymal stem cells.

According to a specific embodiment, the priming and/or differentiation is effected in suspension.

According to a specific embodiment, the culturing for priming is for a period of 2-5 weeks.

According to a specific embodiment, the multipotent stem cells having been obtained by ex vivo maturation of pluripotent stem cells, as described above.

According to a specific embodiment, the ex vivo maturation is effected in suspension the absence of growth factors.

According to a specific embodiment, the pluripotent and/or multipotent stem cells are in an aggregated state optionally of 30-500 μm.

When the cells are primed they can be subjected to terminal differentiation.

Thus there is provided a method of cell differentiation, the method comprising culturing the stem cells having an adipocyte fate while retaining a proliferative phenotype in the presence of fatty acids at a concentration above 100 μM for a time sufficient to allow differentiation to adipocytes.

Priming and differentiation protocols when combined can result in the formation of a microtissue that comprises both adipocytes and muscle cells. Depending on the length of each step, the composition can be controlled in terms of the relative ratio between muscles and fat cells. If the process terminates at the priming step, then the composition would be more muscle-like.

According to a specific embodiment, any of the aggregates or microtissues exhibit organoleptic properties of a native meat product.

As used herein the term “organoleptic properties” refers to the aspects of food, that a consumer experiences via the senses including taste, texture, sight, smell or touch.

For example, adipocytes may be used to confer a non-meat food with a meaty taste and/or texture (e.g., beef or chicken) when cooked or grilled.

Muscle cells or cardiomyocytes may be used to confer a non-meat food with a texture of meat as well as taste (e.g., bitter/metal) of meat.

Erythrocytes may confer a non-meat food with a grilled meat (e.g., beef or chicken) taste as well as meaty color (e.g., beef).

Hepatocytes may confer a non-meat food with meaty color (e.g., beef) as well as enrich the taste of meat.

The term “meat” is meant to encompass any animal flash that is eaten as food (e.g., beef, pork, poultry, fish as well as additional examples which are provided hereinbelow).

In this case, the organoleptic property is that of meat. For instance, when referring to taste, an organoleptic property may be an umami taste.

As used herein “nutritional properties” refer to the composition of meat that is of valuable to the subject feeding on it.

For instance, muscle tissue is very high in protein, containing all of the essential amino acids, and in most cases is a good source of zinc, vitamin B12, selenium, phosphorus, niacin, vitamin B6, choline, riboflavin and iron. Several forms of meat are also high in vitamin K. Muscle tissue is very low in carbohydrates and does not contain dietary fiber. Proteins, vitamins, and minerals are available from almost any source of meat and are generally consistent.

The fat content of meat can vary widely depending on the species and breed of animal, the way in which the animal was raised, including what it was fed, the anatomical part of the body, and the methods of butchering and cooking. Wild animals such as deer are typically leaner than farm animals, leading those concerned about fat content to choose game such as venison. The fatty deposits that exist with the muscle fibers in meats soften meat when it is cooked and improve the flavor through chemical changes initiated through heat that allow the protein and fat molecules to interact. The fat, when cooked with meat, also makes the meat seem juicier. However, the nutritional contribution of the fat is mainly calories as opposed to protein. As fat content rises, the meat's contribution to nutrition declines. In addition, there is cholesterol associated with fat surrounding the meat. The cholesterol is a lipid associated with the kind of saturated fat found in meat.

Table A below compares the nutritional content of several types of meat (per 110 gr). While each kind of meat has about the same content of protein and carbohydrates, there is a very wide range of fat content.

TABLE A

| Source | Calories | Protein (gr) | Carbs (gr) | Fat (gr) |
|---|---|---|---|---|
| Fish | 110-140 | 20-25 | 0 | 1-5 |
| Chicken breast | 160 | 28 | 0 | 7 |
| Lamb | 250 | 30 | 0 | 14 |
| Steak (beef | 210 | 36 | 0 | 7 |
| top round) | | | | |
| Steak (beef | 450 | 25 | 0 | 35 |
| T-bone) | | | | |

Other major nutritional ingredients and their sources are provided hereinbelow:
Vitamin B12, which is mainly found in fish, meat, poultry and dairy products.
Vitamin D, which is found in oily fish, eggs and dairy.
Docosahexaenoic acid (DHA), which is an essential omega-3 fat found in fatty fish.
Heme-iron, which is predominantly found in meat, especially red meat.
Zinc, which is mainly found in animal protein sources, such as beef, pork and lamb.

Hence, the improvement/enhancement in nutritional property can be in any of protein, calories, fat, vitamins, minerals such as listed herein.

As used herein “enhancement”, “increase”, “augmentation” refers to an addition of at least 10%, 15%, 20%, 25%, 30%, 35%, 40%, 45%, 50%, 55%, 60%, 65%, 70%, 75%, 80%, 85%, 90% 95% 100%, 200%, 300%, 400%, 500%, 600%, 700%, 800%, 900% or 1000% (e.g., by weight or by score) of the organoleptic or nutritional property as compared to the same food without the aggregates or microtissues or in which a portion has been substituted with an equivalent amount of the aggregates or microtissues.

Thus, there is provided a food (also referred to as “foodstuff”) comprising the microtissues or aggregates as described herein. The food may comprise meat and/or non-meat portion.

Additionally or alternatively, there is provided a method of producing food, the method comprising combining the microtissues or aggregates, as described herein, with an edible composition for human consumption, such as meat and/or non-meat portion.

According to a specific embodiment, the non-meat is a plant originated substance(s).

According to a specific embodiment, the non-meat is a non-plant originated substances (e.g., minerals, synthetic substance(s)).

According to a specific embodiment, the non-meat is selected from the group consisting of a plant originated substance(s) and non-plant-originated substance(s).

According to a specific embodiment, the foodstuff is a vegetarian foodstuff.

According to a specific embodiment, the foodstuff is a vegan foodstuff.

According to a specific embodiment, the foodstuff comprises a meat substitute or is generally consumed as a meat substitute (plant-based).

According to a specific embodiment, the hybrid foodstuff is free of bodily fluids e.g., saliva, serum, plasma, mucus, urine, feces, tears, milk etc.

The term ‘foodstuff’ refers hereinafter to any substance with food value. The term further refers to the raw material of food before, within the process and after processing, the food product or portions thereof (e.g., the coating of an edible item such as a schnitzel), by-product(s) and end-product (e.g., sausage, ground meat, schnitzel etc.) thereof. In the present invention, there are provided three families of foodstuffs: plant-originated foodstuffs and substances thereof, and aggregates and microtissues, and a foodstuff.

According to an embodiment of the invention the foodstuff is an end article of manufacture (product) to be consumed by a human or non-human subject, or the aggregates and microtissues which are consumed by the food industry in the process of preparing food.

Examples of meat substitutes: natural, traditional and commercially made which are contemplated according to some embodiments of the invention:
Alpro and Provamel, both usually known for their plant milk range, also offer different vegetarian
meat substitutes
Beanfeast
Beyond Meat
Boca Burger
Falafel, a traditional Middle Eastern bean fritter, believed to have been created by ancient Copts as a meat substitute during Lent
Fistulina hepatica, common mushroom known as beefsteak fungus
Ganmodoki, a traditional Japanese tofu based dish similar to veggie burgers
Gardein
Gardenburger
Glamorgan sausage
Goshen Alimentos
Green Slice vegetarian, organic and soy free hot dogs and deli slices
Impossible Foods
Jackfruit, a fruit whose flesh has a similar texture to pulled pork when cooked
Koya-dofu, freeze-dried tofu that has a taste and texture similar to meat when prepared, common in Buddhist vegetarian cuisine
Laetiporus, a mushroom which is also named chicken of the woods
Leaf protein concentrate
LightLife
Linda McCartney Foods
Lyophyllum decastes, mushroom known as fried chicken mushroom
Meat extenders
Meatless
Mock duck
Morningstar Farms
Muscolo di grano (Wheat's muscle), seitan prepared according to an Italian recipe
Nut roast
Oncom
Paneer, for example in such dishes as Paneer tikka
Quorn
Soy protein
Soy pulp, used for veggie burgers and croquettes
Tempeh
Textured vegetable protein
Tofu, not traditionally seen as a meat substitute in Asia, but widely used for that purpose in the
Western hemisphere
Tofurkey
Tofurky
Turtle Island Foods
Vegetarian bacon
Vegetarian hot dog
Veggie burger
Veggie Chicken patty
Veggie Chicken cutlet, breaded (schnitzel)
Veggie Chicken frankfurter (Hot dog)
Viana, one of the largest German vegan food manufacturers, offers a wide range of veggie burgers, croquettes, sausages, minced mock meats, up to vegan döner, vegan gyros and deli slices for sandwiches

### Wheat Gluten

Any of the above examples is an independent embodiment that is not necessarily associated with a specific vendor.

It will be appreciated that food refers to food or feed (animal feeding).

The process of producing food may include any of rising, kneading, extruding, molding, shaping, cooking, stewing, boiling, broiling, baking, frying and any combination of same.

Also provided is a method of providing nutrition to a subject in need thereof. The method comprising providing the subject with a foodstuff as described herein.

According to a specific embodiment, the subject is at risk of nutritional deficiency.

According to a specific embodiment, the subject is a healthy subject (e.g., not suffering from a disease associated with nutrition/absorption).

It will be appreciated that according to a specific embodiment, numerical values indicated are provided under optimal conditions for cell growth of a type and developmental stage as the non-human pluripotent stem cells.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion. Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Methods

Chicken Embryonic Stem Cells (cESCs) Isolation:

Isolation of cESCs from freshly laid chicken (gallus) eggs was performed according to previously published protocol (26). Briefly: Disinfected fertilized eggs shell was removed carefully in order to separate the yolk. Yolk was placed in a 1500 mm dish with the embryo directly placed upward. Small punched ring of sterile 3M paper was placed directly on the embryo to allow dissecting it out. The collected embryo was immersed in cold PBS by taking care to enter the ring vertically to the liquid surface to prevent the embryo from detaching itself. Excess of yolk was removed by gently shaking the embryo. The cleaned embryo was placed in a fresh dish comprising ES medium (below). The collected embryo was plated in a well of a 6-well plate coated with gelatine and MEFs-2 embryos per well. Embryos incubated at 39° C. under 10% CO2. The medium was refreshed every 24 hrs. The eggs were at stage X (day 0).

ES medium composition: DMEM/F-12, 10% Fetal Bovine Serum, 1×MEM Non Essential Amino Acid concentrate, 1 mM Sodium Pyruvate, 10 U/1 U Penicillin/Streptomycin, 2 mM glutamine, 15 uM β-Mercaptoethanol, 5 ng/mL IGF1, 1 ng/mL SCF, 1 ng/mL IL6, 1 ng/mL sIL6 Rα, 20 ng/mL (1,000 U/mL) hLIF.

Adaptation to Growth in Suspension and Scale-Up.

Adaptation of cESC to growth in suspension included propagation of the stem cells on MEFS seeded on gelatin coated plates or by direct cell seeding on gelatin coated plates. Following 2-3 passages, the feeder layer when present was gradually removed. After the establishment of a feeder free population of cESCs, the cells were transferred to non-treated culture plates and placed on an orbital shaker at 75 RPM. Following several passages with daily mechanical breakage of the aggregates (between 30-90 passages) cells began to form loose aggregates of 10-5000 cells/per aggregate, having a size between 30-300 microns. Aggregates were then transferred to a 125 ml flask supplemented with 50 ml ES media. Following several passages (3-5), the cells were transferred to larger flasks (up to 2 L) and allowed to reach a concentration of 2-6×10⁶/ml. Next, several clones were tested in a stirred bioreactor settings to obtain cell lines with a high proliferation rate while maintaining the aggregate's integrity and stem cell characteristics. After several passages, selected clones exhibited the desired morphology, differentiation potential, and reached a doubling time of 8-12 hours per cycle. At this point (following step 7 of FIG. 19) the cells were ready to be inoculated into a stirring bioreactor with factor free media (AMBR 250 and Biostat A/B/B-DCU (2 L, 5 L/10 L respectively) by Sartorius, Eppendorf, Solaris, Thermofisher, New Brunswickm, or Applikon which allows the cells to form microtissues and reach a population density of about 100-300 million cells per ml in Factor-free medium under perfusion conditions. Factor-free medium composition: DMEM 10% Fetal Bovine Serum, 10 U/1 U, Penicillin/Streptomycin, 2 mM glutamine

Bovine ESCs (bESC) Isolation, Adaptation to Suspension and Differentiation:

Bovine ESCs isolation (according to Bogliotti et al 2018):

Fertilized bovine embryo placed on a feeder layer (gamma irradiated MEFS) in 12 well plates and left to adhere and propagate in CTFR-bESC medium (custom made mTESR*) supplemented with 20 ng/ml FGF2 and 2.5 uM IWR1. The Cells passaged every 48 hrs using TrypLE (12563011; Gibco) and re-plated on MEFS in the presence of 10 uM of rho-kinase inhibitor Y-27632. Following 5 passages the use of Y-27632 was depleted. At this stage, cell cryopreserved in cell banks of MEF-dependent bESCs. The adaption to suspension growth was performed as followed. MEF-dependent bESCs were transferred into MEF-free 6-well plates in either CTFR-bESC or mTESR medium supplemented with 20 ng/ml FGF2 and 2.5 uM IWR1 with or without the addition of Y-27632. Cell plates placed in a shaker incubator with gentle stirring (75 RPM), 37 C/5% CO2. The cells were manually pipetted gently every 24 hrs in order to inhibit their adherence to the well surface. Following 3-5 passages cells were transferred to 10 mM plates in a shaker incubator with gentle stirring (75 RPM), 37 C/5% CO2 for 14 days. The medium was replenished every 48 hrs. Following 14 days cells transferred into 125 ml shaker flasks in a total volume of 30 ml mTESR supplemented with 20 ng/ml FGF2 and 2.5 uM IWR1. Cells adapted to these conditions were cryopreserved in cell banks of suspension adapted bESCs. The aggregates selection performed by replenishing the medium every 48 hrs leaving 30-500 um aggregates in flasks. The expansion of bulk and differentiated populations step was performed as follows: Following up to 14 days of a gradual increase in volume Flasks (up to 0.5 L in 2 L flasks) cells were transferred to maturation in Bioreactors. Expansion of bESCs in suspension in monitored stirring bioreactor in factor free media (DMEM (HG)/10-20% FBS, 10 U/1 U, Penicillin/Streptomycin, 2 mM glutamine. For muscle differentiation: growth of cells in large bioreactor with the addition of 5-50 uM OA/LA with or without supplementation of FGF2/50 uM Hydrocortisone/5% horse serum/Gsk inhibitor CHIR99021 (see brown et al. 2014, Adhikari et al., 2019, lian et al 2013) for 7-21 days prior to harvesting. For fat differentiation: growth of cells in large bioreactor with the addition of 200-400 uM OA/LA For 4-7 days prior to harvesting.

### Porcine ESCs Isolation (According to Burrell et al., 2019):

Fertilized porcine embryo placed on a feeder layer (gamma irradiated MEFS) in 12 well plates and left to adhere and propagate in N2B27-3i medium (1:1 ratio of DMEM/F12/Neurobasal medium (21103049; Gibco), 0.5×N2 (17502048; Thermo Fisher), 0.5 XB27 (17504044; Thermo Fisher), 1,000 U/mL human leukemia inhibitory factor (hLIF, L5283; Sigma)], 3i cocktail [30 mM CHIR99021 (GSK3 Inhibition), 40 mM PD0325901 (MEK Inhibitor), 10 mM PD173074 (FGFR3 Inhibitor)], 0.5×GlutaMAX (35050-061; Gibco) supplemented with 0.1 mM b-mercaptoethanol (M3148; Sigma), 1×MEM nonessential amino acids (MEM NEAA, 11140050; Gibco), 0.01% bovine serum albumin (BSA) (A7906; Sigma). Cells passaged every 48 hrs using TrypLE (12563011; Gibco) and replated on MEFS in the presence of 10 uM of rho-kinase inhibitor Y-27632 following 5 passages the use of Y-27632 was depleted (other factors are included in the mTESR medium).

At this stage, cell were cryopreserved in cell banks of MEF-dependent fESCs. Adaptation of pESCs to suspension growth performed as followed: MEF-dependent pESCs were transferred into MEF-free 6-well plates in mTESR medium ((85850; STEMCELL Technologies) with or without the addition of Y-27632. Plates were placed in a shaker incubator with gentle stirring (75 RPM), 37 C/5% CO2. The cells were manually pipetted gently every 24 hrs in order to inhibit their adherence to the well surface. Following 3-5 passages cells were transferred to 10 mM plates in a shaker incubator with gentle stirring (75 RPM), 37 C/5% CO2 for 14 days. The medium was replenished every 48 hrs. Following 14 days, cells were transferred into 125 ml shaker flasks in a total volume of 30 ml mTESR. Cells that adapted to suspension conditions were cryopreserved in cell banks of suspension adapted pESCs. Selection for aggregates growth was performed by replenishing media every 48 hrs leaving large aggregates in flasks.

Expansion of bulk pESCs and differentiated populations performed as follows Following up to 14 days of gradual increase in volume Flasks (up to 0.5 L in 2 L flasks) cells were transferred to maturation in Bioreactors. The expansion of pESCs in suspension in stirring bioreactor was conducted in factor free media (DMEM (HG)/10-20% FBS, 10 U/1 U, Penicillin/Streptomycin, 2 mM glutamine. For muscle differentiation: the growth of cells in a large bioreactor with the addition of 5-50 uM OA/LA with or without supplementation of FGF2/50 uM Hydrocortisone/5% horse serum (see brown et al. 2014, Adhikari et al., 2019) for 7-14 days prior harvesting. For fat differentiation: the growth of cells in a large bioreactor with the addition of 200-400 uM OA/LA for additional 4-7 days prior to harvesting.

Shark, Tuna, Salmon, Herring, Sardine ESCs isolation (according to Hong et al., 1996): In vitro, fertilized embryos were collected following 6-8 hrs (blastula) and trypsinized to a single cell suspension. Cell suspension placed in gelatin coated 6 well dish in ESM media (DMEM(HG) supplemented with 20 mM HEPES (pH 7.4), 10 U/1 U, Penicillin/Streptomycin, 2 mM glutamine, 1 mM Na-pyruvate, 2 nM Na-selenite, 1 mM non-essential amino acids, 50 pM 2-mercaptoethanol, 10 ng/ml bFGF, 10 ng/ml human recombinant LIF, 15% fetal bovine serum and fish serum from trout (FS, 1%). Cells were allowed to propagate and passaged every 48-72 hrs. At this stage cells used for the generation of cell banks of fESCs. Adaptation to suspension growth performed as follows: fESCs were transferred into no coated 6-well plates in ESM1 medium with or without the addition of Y-27632 (mTESR already includes other factors as reported by the manufacturer). Cells plates were placed in a shaker incubator with gentle stirring (75 RPM), 37 C/5% CO2. The cells were manually pipetted gently every 24 hrs in order to inhibit their adherence to the well surface. Following 3-5 passages cells were transferred to 10 mM plates in a shaker incubator with gentle stirring (75 RPM), 37 C/5% CO2 for 14 days. The medium was replenished every 48 hrs. Following 14 days cells transferred into 125 ml shaker flasks in a total volume of 30 ml mTESR. Cells adapted to suspension growth were cryopreserved in cell banks of suspension adapted fESCs.

The selection of aggregates performed by replenishing media every 48 hrs leaving large aggregates in flasks as above. Expansion of bulk and differentiated populations was performed as follows: Following up to 14 days of gradual increase in volume Flasks (up to 0.5 L in 2 L flasks) cells are prepared to maturation in Bioreactors The expansion of fESCs takes place in suspension stirred bioreactor in factor free (DMEM (HG)/10-20% FBS, 10 U/1 U, Penicillin/Streptomycin, 2 mM glutamine. For muscle differentiation: the growth of cells in a large bioreactor with the addition of 5-50 uM OA/LA with or without supplementation of FGF2/50 uM Hydrocortisone/5% horse serum (see brown et al. 2014, Adhikari et al., 2019) for 7-14 days prior harvesting. For fat differentiation: the growth of cells in a large bioreactor with the addition of 200-400 uM OA/LA For 4-7 days prior to harvesting.

Detection of Marker Expression

Aggregates were collected into 15 ml vials and precipitated by centrifugation (800 G/5 min). Following precipitation, cells washed twice with PBS X1. Cells were fixed in 4% PFA for 20 min and washed twice in PBS X1. Following fixation cell's membranes were permeabilized in PBSX1/0.01% Triton X-100) for 15 min. epitope blocking performed by incubating the cells for 1 h in pbsX1/5% goat serum. Following blocking antibodies (Abs) were added to the cells and samples incubated o/n in 4 C and slow shaking. ABs were washed twice from cells and secondary fluorescent AB was added. Cells were incubated for a further 2 hrs with secondary AB and washed three times prior to microscopic observation.

ABs in Use:

| vendor | cat | AB name |
|---|---|---|
| DSHB | PCRP-POU5F1-1D2 | Oc4 |
| ABCAM | ab80892 | Nanog |
| DSHB | MC-631 | SSEA3/4 |
| DSHB | PCRP-LIN28A-1E2 | Lin28 |
| ABCAM | ab16288 | Tra-1-60 |
| DSHB | ens1 | ENS-1 |
| ABCAM | ab34712 | Col2A1 |
| Thermo | PA5-51280 | Col8a2 |
| DSHB | 33-2 | HSPG |
| DSHB | 3H11 | Laminin |
| DSHB | MF20 | MyHC |
| DSHB | CT3 | troponinT |
| DSHB | pax7 | pax7 |
| ABCAM | ab150113 | Goat anti mouse |
| ABCAM | ab150080 | goat anti rabbit |

Telomerase Activity Assay

Telomerase activity was detected in the aggregates using the TRAPeze® Telomerase Detection Kit (S7700 Merck) according to the manufacturer protocol. Briefly: aggregates were isolated and suspended in Chaps lysis buffer. Lysates were added to TS (telomerase substrate) master mix and incubated for 30 min in 37° C. In the next step, samples were taken to PCR amplification using a kit for specific factors. Telomerase activity was detected by gel electrophoresis of the PCR products, where heated samples (95° C.) served as a negative control. The positive control sample provided by the kit (abcam #ab83369).

Alkaline Phosphatase Assay

Detection of Alkaline phosphatase in aggregates was conducted using the Alkaline Phosphatase Assay Kit (abeam #ab83369) according to the manufacturer's protocol. RNA of aggregates was extracted by HyLabs and RNA sequencing was performed by the Weizmann Institute.

RNA Seq.

RNA was extracted from cell pellets using an outsourced service (Hylabs), Sequencing INCPM mRNA-seq. Briefly, total RNA was fragmented followed by reverse transcription and second-strand cDNA synthesis. The double-stranded cDNA was subjected to end repair, a base addition, adapter ligation, and PCR amplification to create libraries. Libraries were evaluated by Qubit and TapeStation. Sequencing libraries were constructed with barcodes to allow multiplexing of 12 samples on a half lane of Illumina NextSeq High output 75 cycles, resulting in ˜20 million single-end 82-bp reads per sample. Bioinformatics: Poly-A/T stretches and Illumina adapters were trimmed from the reads using cutadapt resulting reads shorter than 30 bp were discarded. Reads were mapped to the G. gallus reference genome GRCg6a using STAR, supplied with gene annotations downloaded from Ensembl (and with End To End option and utFilterMismatchNoverLmax was set to 0.04). Expression levels for each gene were quantified using htseq-count, using the gtf above. Differentially expressed genes were identified using DESeq2 with the betaPrior, cooksCutoff, and independentFiltering parameters set to False. Raw P values were adjusted for multiple testing using the procedure of Benjamini and Hochberg. Pipeline was run using snakemake.

Expansion in Factor-Free Conditions (General Description of Step 8b of FIG. 19)

For expansion and maturation the pluripotent aggregates into microtissues with desirable traits for meat production, aggregates were transferred to a factor free media in a stirred bioreactor environment. Under these conditions, the forming microtissues reduced the expression of pluripotency markers (OCT4, Nanog, Lin28) while accumulating expression of members from the collagen family (Col9a2, Col9, Col2a) and the expression of ECM factors (HSPG, CSPG, Laminin). For production of the cultured chicken raw material, the cells were harvested, centrifuged and washed 3 times with PBS. Microtissues were then stored at −20 C until use.

Differentiation of Microtissues into Fat Accumulating Cells (General Description of Step 9a of FIG. 19)

To achieve fat cell differentiation with induced triglycerides synthesis and lipid droplets accumulation microtissue cells were subjected to oleic acid treatment. Oleic acid was either dissolved in 70% ethanol or conjugated to fatty acid-free BSA. Dissolved or conjugated oleic acid was added to factor free media to a final concentration of above 100 uM up to 500 μM (preferably 315 μM). Microtissue were grown with serum for 3-6 days before harvesting.

Differentiation of Microtissues into Muscle Cells (General Description of Step 9b of FIG. 19)

For muscle cell differentiation, microtissues were grown in the presence of 20-50 μM of oleic acid/linoleic acid (or combination of both) for a period of at least 14 days. The medium was refreshed every 72-96 hrs.

Adaptation of Microtissues to Serum-Free Media (General Description for Steps 7-9 of FIG. 19).

Adaptation of microtissues to growth in serum-free media was performed in two parallel methodologies:

Direct selection: Aggregated cells were placed directly in a serum free medium at the concentration of 500K-1M cells/ml. Medium was replenished every 72 hrs. Single cells removed from culture each time the medium was replenished.

Gradual adaptation: aggregated cells were placed in a medium containing DMEM supplemented with 10% FCS alongside 10% serum media mixed with serum free media in ratio of 1:1 (e.g. 250 ml serum/250 ml serum free) the serum free media at the ratio of 1:1. In the following weeks, serum-containing media reduced by 50% after 7 days of culture, until reaching complete drop out of serum free media. Following 7 days the portion of serum media in the mix was reduced by half (e.g. 125 mlserum/375 ml serum free) each week the present inventors gradually reduced the serum media by half until cells were fully adapted to serum free media).

For the experiments using GRO-I/EX-CELL system the medium was prepared according to the manufacturer instructions. In all other experiments lysates were dissolved in the medium at the concentration of 1 gr/L. In all experiments the medium was also supplemented with Ethanolamine (20 ng/L), Insulin (100 ug/L), Selenium (50 ng/L) and Transferrin (55 ug/L). Media also supplemented with 1×MEM NEAA (Biological industries, 01-340-1B).

2 mM L-Alanine/L-Glutamine (Biological industries-03-022-1B) and 1 mM Sodium pyruvate (Biological industries).

Different combinations of yeast/soy lysates were used in order to replace the serum. The lysates used in these experiments were either:

KERRY's: Hypep 1510 (ID: S-2048780, Item: U1-5X99023), SHEFF-VAX PLUS ACF (ID S-2048778, U1-5X00484.K1G), SHEFF-VAX PF ACF (ID:S-2048777, U1-5X01143.K1G), SHEFF-VAX PLUS PF ACF VP (ID: S-2048776, U1-5X01090).

DIFCO: DIFCO's Select Soytone (#15ABP196), Bacto's Yeast Extract (#15ABP197), BBL's Phytone peptone (#15ABP195), BBL's Yeast extract (#15ABP202), Bacto Malt Extract (15ABP201) and Bacto TC yeastolate (#15ABP163)

DIFCO's Select Soytone (#15ABP196), Bacto's Yeast Extract (#15ABP197), BBL's Phytone peptone (#15ABP195), BBL's Yeast extract (#15ABP202), Bacto Malt Extract (15ABP201) and Bacto TC yeastolate (#15ABP163)

IRVINE Scientific (Ultrafiltered Soy Hydrolysate #IR-96857E; Ultrafiltered Yeast Hydrolysate #IR-96863E)

Fatty Acid Composition

Fatty acid composition of cells was analyzed using outsourced service (HUJI).

VOC analysis—Volatile organic compounds analysis was conducted using outsourced service (HUJI)

Nutritional values analysis was conducted using outsourced service (Bactochem)

### Example 1

### Retrieval of cESCs and Expansion

The goal was to establish stem cells derived cell lines capable of prolonged replication and growth in full-suspension while retaining the ability to differentiate into different cell types that will have favorable organoleptic and nutritional properties as well as support various engineering related attributes that allow the support large scale production. As embryonic stem cells (ESCs) hold an indefinite replication capacity and lineal plasticity, the aim was to establish a cell line derived from chicken embryonic stem cells (cESCs) according to a published isolation protocol (26) to obtain the initial cESCs on which further selection, adaptation, and differentiation processes were performed.

The isolation of cESCs was performed based on the protocol of Aubel & Pain, 2013 with some modifications (1). Primary cultures were obtained by isolation of blastoderms from a stage-X embryonic disc of freshly laid chicken eggs. Embryonic cells were then seeded on feeder cells and cultured in stem cell supportive media in a 5% CO2 incubator at 39° C. for 10 days until colonies were easily visible (FIG. 1a).

Colonies were passaged by mechanical dissociation for at least 10 passages. Only clones showing the stability of cell identity and morphology were further used (FIG. 1b).

### Example 2

### Feeder Removal

After successful isolation of cESCs, cells were gradually displaced from the feeder layer and grown for several passages to select for stable feeder-free clones. Notably, after the feeder layer dropout phase, the cells tended to form less compact stem cell colonies composed of large nucleated cells as they were not confined by fibrous cells (FIGS. 1c-e). At this stage, cells exhibited the expected doubling time of about 24 hours per cycle.

### Example 3

### Adaptation to Suspension and Scale-Up Conditions

In order to be able to grow large quantities of cells in a manner that would allow to up-scale the process to the level needed by the food industry, it was necessary to establish a cell line that would be able to grow in full-suspension while retaining self renewal and differentiation abilities with developing new characteristics favorable for food production. To do this, cells were gradually adapted to grow in suspension rather than as adherent cells. When ESCs were moved to suspension conditions, they naturally formed embryonic bodies (EBs) which became more compact as they grew, thus blocking the flow of nutrients and signals from the surface to the inner cell layers, creating varying gradient conditions from the outside of the sphere into the center. This in turn, caused the cells to differentiate and lose their proliferative potential. The aim was therefore to select for cells that would not form compact EBs upon growth in full-suspension (see EB in FIG. 2a) and would still retain the desired stem cell characteristics which underlie the process. To do this, cells were gradually displaced from adhesive surfaces and subtle shaking was imposed. Again, by the continuous selection for a period of about 2-3 months, cells were encouraged to down-regulate different adhesion molecules while expressing others, allowing over time the formation of 3D loose raspberry-like aggregates, with a clear definition of each cell composing the aggregate (FIG. 2F). At this stage, cells have become very stable, and doubling time was reduced to between 18-20 hours per cycle.

Following the adaptation of cells to growth in suspension the present inventors aimed to specify and optimize the growth conditions which are needed to support continued rapid growth in a reproducible manner in a stirred bioreactor environment to ensure the ability of the cells to be suitable for industrial scale-up. For this, several clones were tested for the generation of a cell line that grows as aggregated cells, with a high proliferative rate, in high-velocity stirring (200-400 rpm tip speed) in stirred bioreactors, while maintaining the aggregate's integrity and stem cell characteristics. Following this process of optimization, several cell lines were produced with all favorable characteristics, these cells were named “SMCMC”. These cells exhibited the desired morphology, differentiation potential, and reached a doubling time of 10-12 hours per cycle, with some growing conditions showing 8 hours per cycle. (FIG. 3).

### Example 4

### Characterization of Aggregates Cells-Immortality

The ability of self-renewal of the aggregates (stage 7 of FIG. 19) was tested according to four, well established criteria.

A. Pluripotent marker expression. The expression of canonical stem cell factors such as NANOG, OCT4, and TRA-I-60 were determined by using specific immunofluorescence antibody staining as well as other major pluripotent markers such as SSEA4, LIN28, and ENS-1. Aggregates were found to be positive for all pluripotent markers except for SSEA4, confirming the existence of essential self-renewal factors common to all pluripotent stem cells (FIG. 4).

B. Alkaline phosphatase activity. Alkaline phosphatase staining was found to be positive in aggregates, indicating their proliferative nature (FIG. 5a-b).

C. Long term passaging (>60) with the ability to reach full confluency after each passage/dilution of cells was also tested. Aggregates were grown continuously for at least 180 passages and showed stability of growth and expression of markers.

D. Telomerase activity. Stem cells uniquely express telomerase genes which allow them to maintain chromosomal integrity, therefore, detection of telomerase activity is also considered a key criterion in self-renewal capacity. Aggregates display telomerase activity using TRAPeze telomerase detection kit (FIG. 5c).

### Example 5

### Characterization of Aggregates Cells Stability

Stem cells naturally activate numerous self-renewal pathways and may grow indefinitely if deprived of an adequate environment, either by positive signaling or blockage of differentiation induce stimuli. Aggregates cells exhibit key hallmarks of pluripotent cells, marking them as a stable stem cell line with natural immortality and self renewal capabilities. However, as these cells were established to provide massive yields for food production, the present inventors sought to verify that these cells have not been transformed at some point along the establishment process.

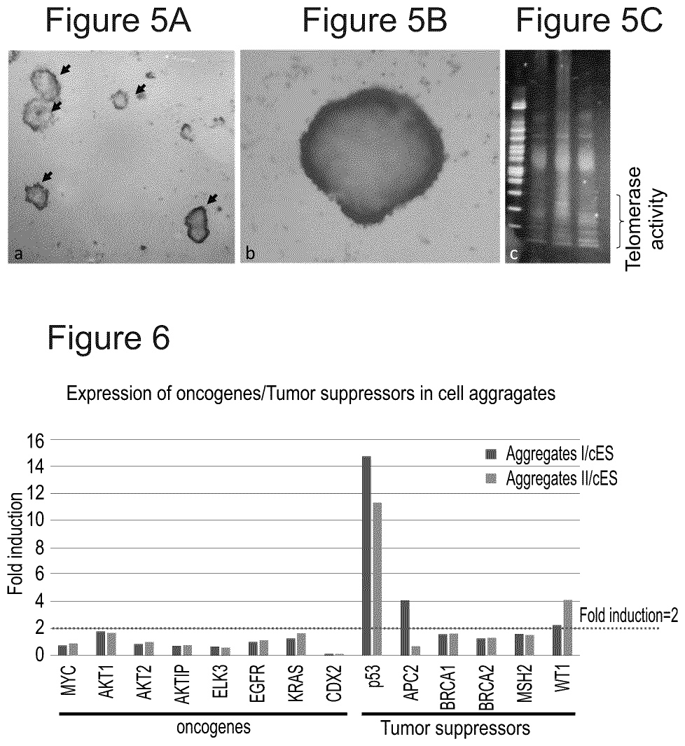

To address this issue, an RNA seq analysis was performed, in order to compare the transcriptional profile of aggregates to the early adherent cESC line, focusing on key cell cycle and metabolic genes. The comparative detailed analysis showed no change in genes related to RNA synthesis and processing, protein translation, binding and activity, all key metabolic and cell cycle processes, were relatively unchanged. Furthermore, no difference in telomerase activity was observed, nor DNA repair mechanisms compared with the early stem cell lines. The expression levels of classic oncogenes such as MYC, AKTs, EGFRs, ELKs, KRASs, and CDX2 were also analyzed and showed no changes in expression levels, with some even being downregulated in aggregates. The expression levels of tumor suppressor genes were also analyzed. Genes such as, p53, APC, BRCA, MSH2, and WT1 displayed similar expression levels between the two cell groups (FIG. 6). When looking at invasive behavior, a reduction in expression levels of cell motility and migration-related genes was observed.

Overall, the present data suggests that the aggregates are naturally immortalized by activation of stem cell regulated gene pathways for self-renewal and there is no apparent evidence for oncogenic transformation.

### Example 6

### Differentiation Abilities of Aggregates Cell Lines

Finally, to confirm that aggregates retain an ability to differentiate into different cell types with possible value for the food industry, several differentiation protocols aimed at producing cells of the mesodermal lineages were tested.

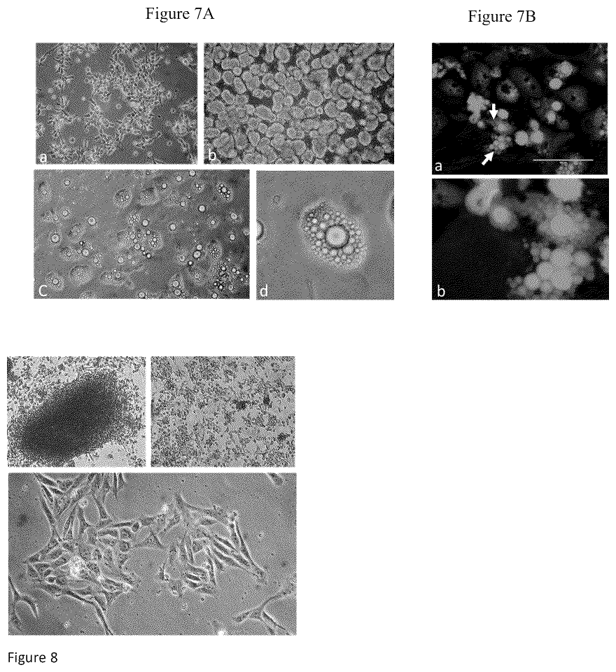

To test aggregates ability to synthesize and store fat, cells were supplemented with adipocyte differentiation media for up to 4 days. Rapid changes in cell morphology could be detected as early as 12 hours after changes in media composition as the cells gradually began to accumulate fat droplets in an enhanced manner (FIG. 7A). BODIPY staining also confirmed the existence of fat droplets within the differentiated aggregates (FIG. 7B).

The aggregates ability to form fibroblasts was also tested as fibroblasts cells are able to secrete different proteins of the connective tissue. The differentiation of aggregates into fibroblasts was carried out using a differentiation medium with fibroblast supportive factors. Cells were supplemented with fibroblast media for 72 hours after which basic growth media was added. A typical epithelial morphology observed within the next 48 hours (FIG. 8). Cells could be kept for over 30 passages and retain their morphology. Fibroblasts could also be frozen and thawed with the ability to maintain fibroblastic phenotype.

Lastly, the ability of aggregates to differentiate into muscle cells was also tested. Aggregates were grown in muscle differentiation media for 72 hours, cells were then moved to a non-stirred environment supplemented with basic growth media. During the next 3 to 8 days, aggregates adhered loosely to the plate, grew in size and rhythmic contractions were observed. Muscle differentiation and contractions could also be achieved in fully floating aggregates as well (FIG. 9) supporting the contributions of these cells to a scalable process for meat production.

### Example 7

### Production Process

### See FIG. 19 as Exemplified but not Limited to Avian Stem Cells

Following the successful isolation and establishment of a chicken stem cell-derived lines which can be expanded and grown in mass quantities and display characteristics favorable for food production, the present inventors have created a cell bank to provide the first stage of the production process with well-defined, unchanged seed trains. The next challenge was using the aggregates platform to produce raw material with chicken flavors in a scalable process.

For this task, careful media tailoring, mechanical process development, and regular sensoric tests were carried out.

Seed Train Expansion Stage

The first step of the production process would include thawing a new batch of aggregates from the cell bank. These cells would then be expanded rapidly to the appropriate concentration to supply the downstream bioreactors of the production where the aggregates will transform into micro-tissues and continue to proliferate and change their identity to suit the food industry needs.

Micro-tissue Forming Stage

Several attributes must be present in order to achieve the desired chicken flavors. These include fat quantity and composition, expression of different proteins, and the presence of specific sugars. By manipulation of the maturation medium with careful tasting and chemical analyses, the present inventors were successful in developing a process for obtaining micro-tissues grown in a full suspension that have an enhanced savory chicken flavor. The maturation medium contains no added growth factors, causing the cells to lose key pluripotent markers, while gaining mesodermal properties, including specific markers, and elevated expression of connective tissue proteins. Due to their early identity and specific culturing conditions in the bioreactor, these microtissues are still able to proliferate rapidly and extensively and be expanded for at least 150 population doublings.

Control of these properties is done by defined set-points of the bioreactor run parameters, such as agitation inside the bioreactor and gassing with tip speed ranging from 0.25-1.70 m/sec with optimal tip speed of 0.85 m/sec.

### Example 8

### Adaptation of Aggregates and Microtissues to Grow in Serum Free Media Micro-Tissues to Growth in Serum Free Media

The key challenge in culture meat production is related to the media being used to culture the cells. Tissue culture media were originally designed for other purposes (e.g. research, pharmaceutical, clinical production and more) and therefore carry several caveats from the perspective of culture meat production. The cost of the culture media is generally the biggest economic burden on cultured meat production. Furthermore, most culture media available contain fetal calf (or other animal originated) serum (FCS). The use of FCS harbors the inherent flaw of being undefined (in terms of chemical composition) with high variability between batches. Moreover, the use of such material in culture meat production will most likely alienate public acceptance due to the problematic way it is produced. In order to overcome these obstacles the present inventors set out to adapt the aggregates and microtissues to grow in serum free media, without compromising the quality, safety, and the unique characteristics of the microtissues. In order to achieve this, microtissues were adapted to grow in several combinations of serum free media. As an initial stage the present inventors adapted microtissues to the commercially available serum free system GRO-I/EX-CELL manufactured by SIGMA-Merck. The system is based on cell growth in basal medium (GRO-I) supplemented with chemically defined yeast/plant hydrolysate (EX-CELL) as a substitute agent to the FCS. The adaptation process took place by either complete drop of the serum from cultured media or by gradual depletion of serum concentration over time either as a preliminary stage to seeding in bioreactors (7b) or directly upon seeding cells in stirred bioreactor (FIG. 19, stage 8B). In both cases cells (aggregates or microtissues) were able to adapt the growth in serum free media (in flasks and bioreactor). About 14 days the cells were adapted to the new media, the original 12 hours doubling time was restored and retained a high level of cell viability (FIG. 10). In a similar manner the present inventors were also able to adapt the cells to grow in other combinations of basal media supplemented with yeast, plant peptones and hydrolysates. Among the mixes which were used successfully for adaptation of cells to serum-free media the present inventors could identify: DMEM (high glucose) supplemented with the Ex-Cell lysate, DMEM/F12 supplemented with Ex-Cell lysates, DMEM (low glucose) supplemented with Ex-Cell lysate, DMEM (high glucose) supplemented with combination of soy and yeast lysates manufactured by KERRY group. Lysates that tested successfully in this process were either a combination of all or part of these four products were: Hypep 1510 (ID: S-2048780, Item: U1-5X99023), SHEFF-VAX PLUS ACF(ID S-2048778, U1-5X00484.K1G), SHEFF-VAX PF ACF (ID:S-2048777, U1-5X01143.K1G), SHEFF-VAX PLUS PF ACF VP (ID: S-2048776, U1-5X01090). The present inventors also successfully adapted the microtissues to combinations of routinely used basal media (DMEM HG/LG, DMEM/F12, RPMI 1640) with Soy and yeast hydrolases manufactured by FUJIFILM-IRVINE Scientific (Ultrafiltered Soy Hydrolysate #IR-96857E; Ultrafiltered Yeast Hydrolysate #IR-96863E). In addition, cells were also adapted to grow in basal media (DMEM HG/LG, DMEM/F12, RPMI 1640) supplemented with different combinations of DIFCO's Select Soytone (#15ABP196), Bacto's Yeast Extract (#15ABP197), BBL's Phytone peptone (#15ABP195), BBL's Yeast extract (#15ABP202), Bacto Malt Extract (15ABP201) and Bacto TC yeastolate (#15ABP163). For the purpose of the latter processes, Yeast originated lysates were combined with either one or several plant originated lysates. In all experiments the media also supplemented with Ethanolamine (20 ng/L), Insulin (100 ug/L), Selenium (50 ng/L) and Transferrin (55 ug/L). Media also supplemented with 1×MEM NEAA (Biological industries, 01-340-1B), 2 mM L-Alanine/L-Glutamine (Biological industries-03-022-11B) and 1 mM Sodium pyruvate (Biological industries). The results presented herein emphasize the ability of microtissues to adapt multiple combinations based on basal media supplemented with soy and plant hydrolysates as a substitution to serum in cultured media.

### Example 9

### Molecular Characterization of Micro-Tissues

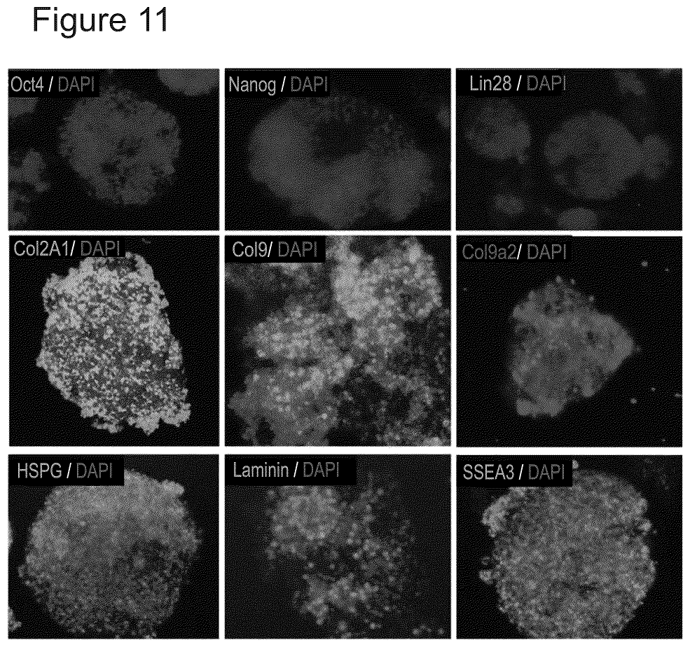

During the micro-tissue forming stage (8b of FIG. 19), the cells lose some of their pluripotent markers, such as NANOG, OCT4, LIN28, but not SSEA3, (FIG. 11) and gain some mesodermal properties such as fat storage and secretion of extracellular matrix proteins (ECM) that surround each aggregate and form a specific niche that creates the micro-tissues. These ECM proteins also contribute to the micro-tissues developing their enhanced chicken flavor and ability to form a mass when cooked together.

The micro-tissues that are derived from the microtissues cells show different mesodermal characteristics. These include structural proteins like Fibronectins, Laminins, and Collagenes which make up the ECM and organize cells during tissue development. Also, the cells express SNAI1, which is a master regulator of formation and maintenance of embryonic mesoderm. Finally, the cells have the ability to synthesize and store triglycerides. Molecular functional analysis and KEGG analysis also revealed that micro-tissues are enriched for the Wnt canonical and non-canonical related factors. Wnt signaling is tightly connected to the acquisition of mesenchymal identity as was suggested by gene expression analysis.

One important factor in the ability of the cell culture to recreate the experience of meat consumption relies on the expression of certain molecules with a significant contribution to the texture and taste of these cells. A major contribution to these aspects comes from the presence of ECM molecules anchored to the cell's membrane or secreted by the cells. In fact, most of the variability between textures of different muscle tissues, is a direct consequence of the collagens content in its fibers (32). The collagen superfamily consists of 28 members with numerous derivatives. Collagens attach to the fibrils and provide both mechanical support as well as numerous biological activities via interactions with other ECM and functional factors. Notably, in the RNA-seq analysis, upregulation of collagen-family members and collagen related proteins such as Col2A, Col8A2, and Col9A2 were identified. Furthermore, the RNA seq analysis revealed a significant enrichment with additional ECM and cell adhesion molecules. This finding holds specific significance since collagen and proteoglycan interactions, such as HSPGS, and CSPG, and Laminin, all of which upregulate in the micro-tissues, are at the base of the cell's matrix organization, structure, and function.

In order to confirm the expression of key ECM factors, an ECM-related antibody-specific screen was performed. Results of the screen confirmed the enrichment of distinct collagens and other ECM molecules in micro-tissues (FIG. 11 exemplifying step 8b of FIG. 19).

### Example 10

### Differentiation of Microtissue for Enhancement of Fat and Muscle Cells

In order to control the exact parameters of the final meat product, the next step was to develop a process, which allows to direct and tune the percentage of differentiation into either fat, muscle, or connective tissue. For this purpose a process was developed, which includes the culturing several cell lines with different combinations of fatty acids in different concentrations.

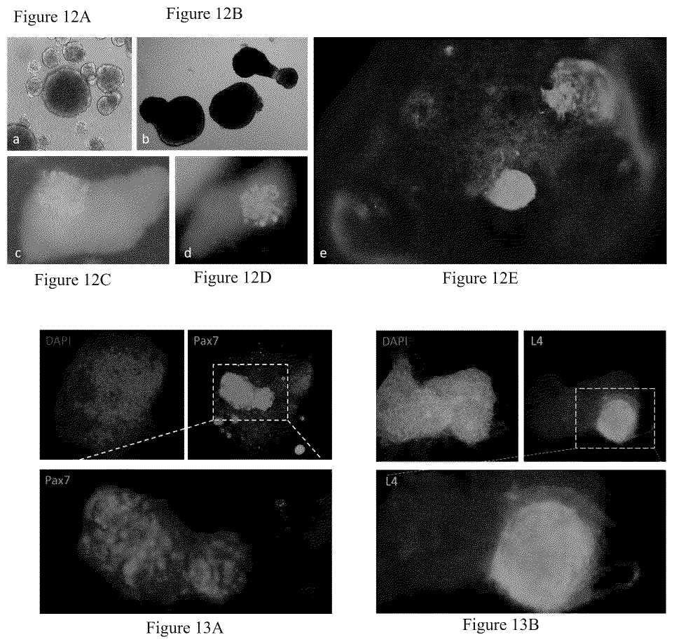

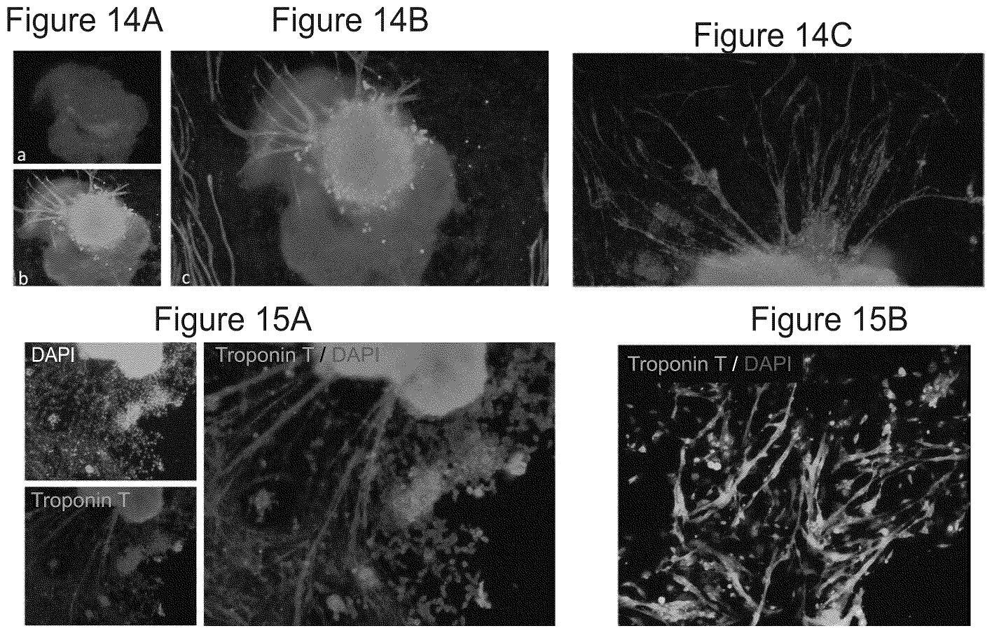

The treatments included growing the cells with differentiation media supplemented with either 20 um-300 um of Oleic acid (OA) or Linoleic acid (LA) and a combination of the two. The findings revealed that the prolonged growth with fatty acids (FA) concentrations below 50 uM allow the propagation of the cells. The culturing of micro-tissues in low concentrations of fatty acids (FAs) leads the micro-tissues to develop distinguishable protrusions in a highly reproducible manner within 96 hours (FIGS. 12A-E). Further analyses revealed that onset of muscle differentiation occurs as early as 4-7 days after introducing it to the FA, prior to the emergence of protrusions by immuno-staining using MyHC antibody (FIG. 12A-E). The cells that underwent priming, after maturation, began to express early markers of muscle differentiation as early as 4-7 days, however, as the cells are still proliferative the stage could be extended to weeks e.g., 3 weeks. FIGS. 12A-E and 13A-B are taken from stage 9b, showing the onset of muscle differentiation that begins with the addition of GFs. Thus, when this stage was prolonged more muscle cells were evidence (FIG. 14A-C) or to the addition of higher concentrations of FAs was used to enhance fat accumulation. MyHC staining, FIG. 12A-E, shows muscle differentiation. In order to confirm the presence of muscle progenitors on top of micro-tissue protrusions, the present inventors also stained the cells using AB against muscle progenitor markers Pax7 and L4. In both cases cells within the protrusion stained positive (FIG. 13A-B). Further staining of micro-tissues cultured in the presence of FA in low doses was able to confirm muscle maturation on top of micro-tissue while still in suspension growth conditions (MyHC staining, FIG. 14A-C). Notably, the existence of mature contracting muscles on top of micro-tissues was demonstrated by documenting the synchronic contractions of micro-tissues, growing in the presence of FA for 14 days (data not shown). In addition, the ability of muscle progenitors within micro-tissue to terminally differentiate into mature muscle was determined by Troponin-T stainings of both suspension and adherent conditions (FIG. 15A-B).

Alongside the emergence of muscle progenitor sites in different locations of the Micro-tissues. FA concentrations of 50-200 uM allow fat storage within the micro-tissue, excluding the areas in which muscle differentiation occurs. Culturing the cells in higher amounts of FA (>200 uM) will lead to a global and terminal differentiation of micro-tissues into fat accumulating tissues and will eventually bring the culture into proliferation arrest.

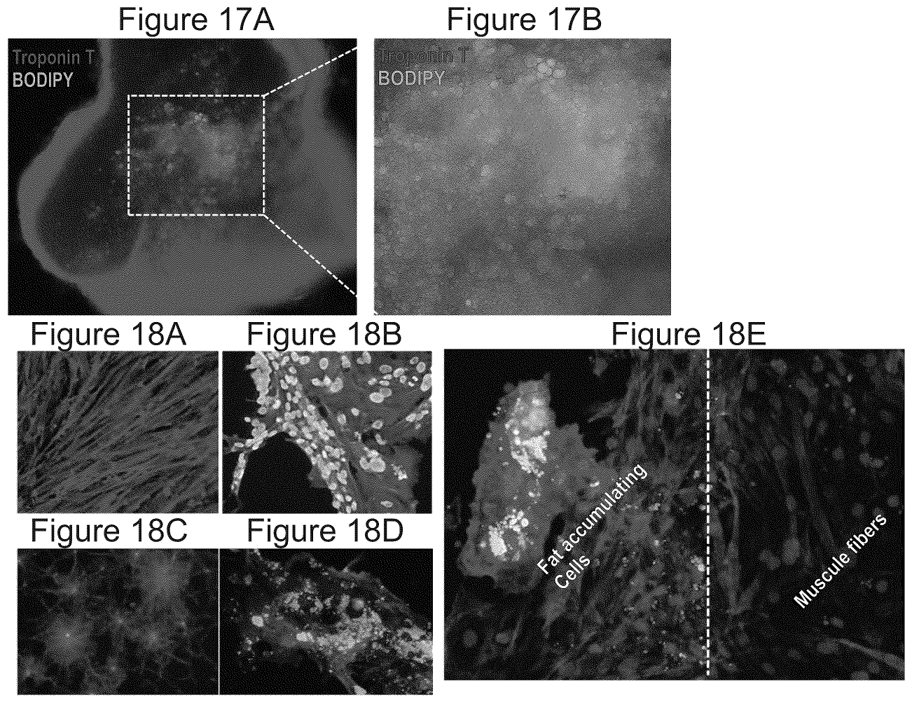

The present inventors have found that prolonged treatment with low doses of either Oleic or Linoleic acids or combined will not only spark the muscle differentiation, but at the same time drive part of the micro-tissue cells to accumulate fat (step 9b of FIG. 19). Microtissues were double stained with TroponinT AB alongside lipid staining (Bodipy) and showed remarkable mutual exclusive populations within micro-tissue differentiated into either fat or muscle lineages (FIG. 16). Furthermore, transferring the micro-tissues into adherent growth conditions led to differentiation of both mature fat cells as well as mature prolonged muscle fiber (see FIG. 17A-B). The two populations can be easily distinguished based on cytoskeletal organization (FIG. 18A-E). Further staining for fat accumulation clearly shows the ability of micro-tissues to differentiate in parallel to both fat and muscle tissues (FIG. 18A-E). These characteristics make the differentiation process of the micro-tissue highly preferable for cultured meat production since they allow to control the characteristics of the raw material by changing the percentages of fat and proteins by demand.

### Example 11

### Organoleptic and Nutritional Properties of Micro-Tissues

During cooking, different chemical reactions take place between different components of the food. One of the most important and complicated of these reactions is the Maillard reaction where amino acids react with reducing sugars to form new molecular compounds responsible for the deep meaty flavor and aromas.

Another major factor controlling flavor delivery and the unique aromas each meat produces during cooking is the fat content and composition.

The medium composition (factor free medium and differentiation media supplemented with FAs) allows for the formation of the desired components within the micro-tissues to produce similar amino acids and fatty acids profiles for the development of chicken flavors as visible from the amino acid composition analysis as well as the fatty acid profiling by gas-chromatography mass-spectrometry analysis. (Tables 1-3).

Multiple tasting of the product was performed by expert tasters and chefs, which reported a tasting experience of enhanced, tasteful chicken flavors. In order to show this in an analytical manner, a preliminary volatile compounds (VOC) analysis was performed on the cooked product and cooked chicken breast. Results showed a highly parallel pattern of VOC release, indicating the development of similar aromas between the two samples (FIG. 19).

Finally, the present inventors wanted to verify the nutritional values of the product. For all the major components of food products were analyzed such as moisture, carbohydrates, proteins, fats, and minerals. All values have shown that the overall nutritional composition of the micro-tissues is greatly comparable with that of chicken meat (Table 3).

TABLE 1
comparison between amino acid profile of chicken breast
sample vs. micro-tissue profile. Analysis revealed a
similar profile between samples (step 8b of FIG. 19).

| Amino Acid | Chicken Breast | SM microtissue |
|---|---|---|
| Cysteic acid | 1.40 | 1.68 |
| Aspartic acid | 9.72 | 9.64 |
| Methionine sulfon | 2.80 | 1.99 |
| Threonine | 4.40 | 4.72 |
| Serine | 3.72 | 4.72 |
| Glutamic acid | 15.47 | 14.26 |
| Proline | 3.53 | 3.35 |
| Glycine | 5.27 | 5.45 |
| Alanine | 6.00 | 6.18 |
| Va,ine | 5.51 | 5.87 |
| Isoleucine | 4.98 | 4.40 |
| Leucine | 8.27 | 9.22 |
| Tyrosine | 3.58 | 3.98 |
| Phenylalanine | 4.30 | 4.93 |
| Lysine | 9.33 | 8.91 |
| Histidine | 4.06 | 2.73 |
| Arginie | 7.64 | 7.97 |

TABLE 2
Comparison of fatty acid profiles between micro-tissues
vs. chicken breast tissue (step 8b of FIG. 19).

| | | Chicken | SM mi- |
|---|---|---|---|
| Fatty acid | | breast | crotissue |
| Palmitic acid | C16:0 | 20.68 | 22.40 |
| Palmitoleic acid | C16:1 (cis-9) | 3.42 | 5.53 |
| Stearic acid | C:18:0 | 9.15 | 11.38 |
| Oleic acid | C18:1 (cis-9) | 32.47 | 33.13 |
| Linoleic acid | C18:2 (cis-9,12) | 18.74 | 0.14 |
| Eicosanoic acid | C20:1 (cis-11) | 0.41 | 1.94 |
| Arachidonic acid | C20:4 | 3.27 | 0.92 |
| Eicosapentenoic acid | c20:5(cis-5,8,11,14,17) | 0.00 | 0.00 |
| Erucic acid | C22:1 (cis-13) | 0.00 | 0.31 |
| Docosahexenoic acid | C22:6 (cis-4,7,10,13,16,19) | 0.30 | 0.74 |

TABLE 3
Nutritional values analysis of micro-tissues vs. commercial
chicken breast sample indicates highly comparable
values between samples. (step 8b of FIG. 19).

| | SM | Chicken | |
|---|---|---|---|
| | Micro- | Breast | |
| | tissue | (no fat) | Units |
| Ash | 1.946078431 | 1.5 | (g/100 g) |
| Caloric Value | 103.1616176 | 95 | (KCal/100 g) |
| Carbohydrates | 0.5838235294 | 0.4 | (g/100 g) |
| Est | 3.133186275 | 1.12 | (g/100 g) |
| Moisture | 76.18 | 76.18 | (g/100 g) |
| Protein | 18.15691176 | 20.83 | (g/100 g) |
| Ca | 65 | 55 | mg/kg |
| Fe | 3 | 3 | mg/kg |
| Na | 2452 | 1715 | mg/kg |
| P | 2281 | 2561 | mg/kg |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of producing a microtissue comprising one or more cell types, the method comprising:
(a) forming in suspension, without matrix adherence, aggregates comprising non-human pluripotent stem cells, said non-human pluripotent stem cells of said aggregates exhibiting a doubling time of 10-20 hours in an undifferentiated manner for more than 60 passages, capable of differentiating into muscle, fat and connective tissue upon differentiation induction, growing in the presence of growth factors, exhibiting cell to cell adhesion lower than that of embryoid bodies (EBs) as determined by reduced expression of adhesion molecules selected from the group consisting of COL6A2, CD44, COL6A1, ANXA1, ANXA2 and S100A11 as compared to said EBs; and
(b) transferring the aggregates to a bioreactor for growth in suspension without matrix adherence in the absence of growth factors to thereby produce the microtissue.

2. A method of producing a microtissue comprising one or more cell types, the method comprising:
(a) providing a suspension culture which comprises aggregates comprising non-human pluripotent stem cells, said non-human pluripotent stem cells of said aggregates exhibiting a doubling time of 10-20 hours in an undifferentiated manner for more than 60 passages, capable of differentiating into muscle, fat and connective tissue upon differentiation induction, growing in the presence of growth factors, exhibiting cell to cell adhesion lower than that of embryoid bodies (EBs) as determined by reduced expression of adhesion molecules selected from the group consisting of COL6A2, CD44, COL6A1, ANXA1, ANXA2 and S100A11 as compared to said EBs; and
(b) transferring the aggregates to a bioreactor for growth in suspension without matrix adherence in the absence of growth factors to thereby produce the microtissue.

3. A method of producing a microtissue comprising one or more cell types of interest, the method comprising:
(a) producing the microtissue according to claim 1; and
(b) subjecting said microtissue to differentiation conditions in suspension to thereby produce the microtissue comprising one or more cell types of interest.

4. The method of claim 1, wherein numerical values indicated are provided under optimal conditions for cell growth of a type and developmental stage as said non-human pluripotent stem cells.

5. The method of claim 1, wherein said non-human pluripotent stem cells are selected from the group of avian pluripotent stem cells, bovine pluripotent stem cells, porcine pluripotent stem cells, goat pluripotent stem cells, sheep pluripotent stem cells, shrimp pluripotent stem cells and fish pluripotent stem cells.

6. The method of claim 1, wherein said microtissue is 30-500 μm in diameter.

7. The method of claim 1, wherein said non-human pluripotent stem cells of said aggregates exhibit alkaline phosphatase expression, exhibit telomerase gene expression and/or are SSEA4−, LIN28+, ENS-1+, NANOG+, OCT4,+ and TRA-1-60+.

8. The method of claim 1, wherein said one or more cell types are selected from the group consisting of a muscle cell, a fat cell and a connective tissue cell.

9. The method of claim 1, wherein said one or more cell types comprise a fat cell and a muscle cell.

10. The method of claim 1, wherein said matrix adherence is selected from feeder cells and a native or synthetic matrix molecule.

11. The method of claim 10, wherein said matrix molecule comprises gelatin.

12. The method of claim 1, wherein each step of the method is devoid of animal components other than said non-human pluripotent stem cells.

13. The method of claim 1, wherein said aggregates exhibit about the same gene expression as that of a stem cell line from which they are derived, excluding expression levels of cell motility and migration-related genes.

14. The method of claim 1, wherein said microtissue is NANOG-OCT4− LIN28−, SSEA3+.

15. The method of claim 3, wherein said subjecting said microtissue to differentiation conditions comprises priming said microtissues in the presence of fatty acids at a concentration not exceeding 100 μM for a time sufficient to obtain muscle cells and stem cells having an adipocyte fate while retaining a proliferative phenotype, optionally followed by differentiating in the presence of fatty acids at a concentration of 100 uM or above.

16. The method of claim 1, wherein said doubling time is of no more than 12 hours.

17. The method of claim 2, wherein said doubling time is of no more than 12 hours.

18. A microtissue obtainable according to the method of claim 1.

19. A microtissue comprising one or more cell types, said microtissue being 30-500 μm in diameter, wherein cells of said microtissue are NANOG-OCT4− LIN28−, SSEA3+, and wherein optionally said one or more cell types comprise a fat cell and a muscle cell.

20. A food comprising the microtissue of claim 19.

21. A method of producing food, the method comprising combining microtissue of claim 19 with an edible composition for human consumption.
